(19) United States
(12) Patent Application Publication
(10) Pub. No.: US 2020/0199041 A1
(43) Pub. Date: Jun. 25, 2020

(54) PROCESSES FOR CONVERTING NAPHTHA TO DISTILLATE PRODUCTS

(71) Applicant: ExxonMobil Research and Engineering Company, Annandale, NJ (US)

(72) Inventors: Aaron Sattler, Annandale, NJ (US); Kun Wang, Bridgewater, NJ (US); Michele Paccagnini, Randolph, NJ (US); Suzzy C. Ho, Princeton, NJ (US); Jonathan E. Mitchell, Easton, PA (US)

(21) Appl. No.: 16/709,111

(22) Filed: Dec. 10, 2019

Related U.S. Application Data
(60) Provisional application No. 62/783,490, filed on Dec. 21, 2018.

(51) Int. Cl. C07C 5/03; C07C 5/333; C07C 6/06

(52) U.S. Cl. CPC C07C 5/03; C07C 5/3332; C07C 2531/24; C07C 2523/72; C07C 2601/16; C07C 6/06

(57) ABSTRACT

The present disclosure provides processes to convert heavy hydrocarbons to light distillates. The present disclosure further provides compositions including light distillates. In an embodiment, a process for upgrading a hydrocarbon feed includes dehydrogenating a C₃-C₅₀ cyclic alkane and an C₂-C₅₀ acyclic alkane in the presence of a dehydrogenation catalyst to form a C₃-C₅₀ cyclic olefin and a C₂-C₅₀ acyclic olefin. The process includes reacting the C₃-C₅₀ cyclic olefin and the C₂-C₅₀ acyclic olefin in the presence of a group 6 or group 8 transition metal catalysts to form a C₅-C₂₀₀ olefin. The process further includes hydrogenating the C₅-C₂₀₀ olefin in the presence of a hydrogenation catalyst to form a C₅-C₂₀₀ hydrogenated product. Processes of the present disclosure may further include hydroisomerizing the C₅-C₂₀₀ hydrogenated product in the presence of a hydroisomerization catalyst to form a C₅-C₂₀₀ hydroisomerized product.

### CROSS-REFERENCE TO RELATED APPLICATIONS

This application claims priority to U.S. Provisional Application Ser. No. 62/783,490 filed Dec. 21, 2018, which is herein incorporated by reference in its entirety.

### FIELD

The present disclosure provides processes to convert naphtha range hydrocarbons to distillates. The present disclosure further provides compositions including distillates.

### BACKGROUND

As the production of shale and tight oils is increasing in the United States of America, Natural Gas Liquids (“NGL”) and naphtha are becoming increasingly abundant. Ethane to light naphtha range paraffins are largely fed to steam crackers or dehydrogenated to make olefins. For example, ethane is steam-cracked to make ethylene, and light naphtha (b.p. 60° F.-160° F.) is steam cracked to make ethylene, propylene, and small volumes of dienes. Short-chain alkanes (e.g., C₂ to C₅ alkanes) can also be converted to their corresponding olefin using dehydrogenation technologies. Dehydrogenation of short-chain alkanes (e.g., C₂ to C₅) commonly uses one of two types of catalysts:

platinum-based catalyst(s) or chromium oxide catalyst(s). The dehydrogenation process is typically carried out at temperatures >450° C., and under ambient or sub-ambient pressure. To manage the frequency of catalyst regeneration due to coking, reactors such as moving-bed, cyclic swing-bed, or fluidized bed reactors are employed. On the other hand, heavy naphtha (b.p. 160° F.-360° F.) is typically fed to catalytic reformers in order to produce aromatics (as chemicals or high octane gasoline blend), and hydrogen, but no catalyst/process that selectively dehydrogenates naphthenes to mono-olefins has been described.

As the reformers reach capacity, coupled with the limited growth in demand for aromatics and gasoline, there is a continuous need to convert heavy naphtha, particularly heavy virgin naphtha (HVN), to large volume, higher value products. Furthermore, global transportation fuels outlook suggests that the long-term demand for automotive gas (e.g., gasoline) will decrease, while the demand for octane is expected to grow with the increasing use of high-compression engines. Conversely, global fast growing demands for distillate (e.g., jet, diesel) favors the conversion of heavy naphtha (low-octane gasoline; e.g., Research Octane Number (“RON”) and Motor Octane Number (“MON”) for cyclohexane are 83.0 and 77.2, respectively; RON and MON for n-heptane are zero) to distillate range liquids.

Furthermore, the excess in supply of light alkanes and olefins due to shale gas and hydraulic fracturing (also referred to as “fracking”), in addition to traditional light cuts (e.g., C₅ of the Fluid Catalytic Cracking, “FCC”), has limited new uses of these products. Hence, growing the molecular weight of light alkanes and olefins into fuel/lubricant ranges would be valuable, particularly due to the lower value of light alkanes, and the higher value of fuels, and lubricant range hydrocarbons.

Therefore, there remains a need for processes that provide a highly efficient and economical conversion of heavy hydrocarbons to light distillates and/or mid-distillates, such as distillate range liquids, under mild conditions. Furthermore, there is a need for processes to convert heavy naphtha, particularly naphthene-rich heavy virgin naphtha, to distillate range products.

References for citing in an Information Disclosure Statement (37 CFR 1.97(h)): Sattler, J. J. H. B.; Ruiz-Martinez, J.; Santillan-Jimenez, E.; Weckhuysen, B. M. “Catalytic Dehydrogenation of Light Alkanes on Metals and Metal Oxides”, Chemical Reviews (2014), 114, 10613-10653; Patton, P. A.; Lillya, C. P.; McCarthy, T. J. Macromolecules (1986), 19, 1266-1268; U.S. Pat. No. 3,575,947; Dobereiner, G. E.; Erdogan, G.; Larsen, C. R.; Grotjahn, D. B.; Schrock, R. R. ACS Catal. (2014), 4, 3069-3076; U.S. Pub. No. 2007/0083066 A1; Lwin, S.; Wachs, I. E. “Olefin Metathesis by Supported Metal Oxide Catalysts”, ACS Catal. (2014), 4, 2505-2520; U.S. Pat. No. 9,181,360 B2.

### SUMMARY

In an embodiment, a process for upgrading a hydrocarbon feed includes dehydrogenating a C₃-C₅₀ cyclic alkane (e.g., a naphthene), where the ring size is 3 to 8 carbons, and a C₂-C₅₀ acyclic alkane in the presence of a dehydrogenation catalyst to form a C₃-C₅₀ cyclic olefin and a C₂-C₅₀ acyclic olefin. The process includes reacting one or more C₃-C₅₀ cyclic olefin and the C₂-C₅₀ acyclic olefin via olefin metathesis in the presence of a transition metal catalyst, such as a group 6, 7, or 8 transition metal catalyst, to form larger molecules that contain 2 or more carbon-carbon double bonds (e.g., C₅-C₂₀₀ olefins, such as C₅-C₁₀₀ olefins). The process further includes hydrogenating the C₅-C₂₀₀ olefins in the presence of a hydrogenation catalyst to form a C₅-C₂₀₀ hydrogenated product (e.g., alkane).

In another embodiment, a process for upgrading a hydrocarbon feed includes dehydrogenating a C₃-C₅₀ cyclic alkane and a C₂-C₅₀ acyclic alkane in the presence of a dehydrogenation catalyst to form a C₃-C₅₀ cyclic olefin and a C₂-C₅₀ acyclic olefin. The process includes reacting the C₃-C₅₀ cyclic olefin and the C₂-C₅₀ acyclic olefin in the presence of a transition metal catalyst, such as a group 6, 7, or 8 transition metal catalyst, to form larger molecules that contain two or more carbon-carbon double bonds (e.g., C₅-C₂₀₀ olefins, such as C₅-C₁₀₀ olefins). The process further includes hydrogenating the C₅-C₂₀₀ olefins in the presence of a hydrogenation catalyst to form a C₅-C₂₀₀ hydrogenated product (e.g. alkane). The process further includes hydroisomerizing the C₅-C₂₀₀ hydrogenated product in the presence of a hydroisomerization catalyst to form a C₅-C₂₀₀ hydroisomerized product.

### BRIEF DESCRIPTION OF THE DRAWING

FIG. 1 is a gas chromatogram of an n-heptane feed and dehydrogenation products using CuO, according to one embodiment.

FIG. 2 is a gas chromatogram of a cyclohexane feed and dehydrogenation products using CuO, according to one embodiment.

FIG. 3 is a gas chromatogram of products of a ring opening cross-metathesis between trans-4-octene and cyclopentene, according to one embodiment.

FIG. 4 is a gas chromatogram of products of a ring opening cross-metathesis between trans-4-octene and cyclohexene, according to one embodiment.

FIG. 5 is a gas chromatogram of products of a ring opening cross-metathesis between 1-heptene (and dodecene) and cyclohexene, according to one embodiment.

FIG. 6 is a gas chromatogram of products of a ring opening cross-metathesis between a mixture of pentenes with homogeneous Grubbs 2^{nd} generation catalyst, according to one embodiment.

FIG. 7 is a gas chromatogram of products of a ring opening cross-metathesis between a mixture of pentenes with heterogeneous Re catalyst, according to one embodiment.

FIG. 8 is ¹H NMR spectra illustrating lubrication range products after hydroisomerization, according to one embodiment.

FIG. 9 is ¹H NMR spectra illustrating lubrication range products after hydroisomerization between cyclooctene and 4-octene, according to one embodiment.

FIG. 10 is a gas chromatogram of products of a ring opening cross-metathesis between methyl-cyclopentene and 4-octene, according to one embodiment.

### DETAILED DESCRIPTION

The present disclosure provides C₅-C₂₀₀ hydrocarbon products, such as C₅-C₁₀₀ hydrocarbon products and processes for making such C₅-C₂₀₀ hydrocarbon products, such as C₅-C₁₀₀ hydrocarbon products. Processes include converting hydrocarbons (such as heavy naphtha, including paraffins and/or naphthene-rich heavy virgin naphtha, such as C₃-C₅₀ cyclic and/or C₂-C₅₀ acyclic alkanes (e.g., linear and or branched acyclic alkanes) to light distillates. In at least one embodiment, processes include a dehydrogenation stage, a cross-metathesis stage, and a hydrogenation stage to produce polyolefin products.

Catalyst systems used for processes of the present disclosure include one or more alkane dehydrogenation catalyst, ring-opening metathesis catalyst, and/or polymerization catalyst, olefin hydrogenation catalyst, and an optional support. In one aspect, a light distillate product includes the one or more C₅-C₂₀₀ hydrocarbon product(s), such as the one or more C₅-C₁₀₀ hydrocarbon product(s). The light distillate product may be blended with one or more other components (e.g., additives) to produce, for example, a fuel composition (e.g., higher value diesel (cetane)), waxes, lubricant range products, and base stocks.

The present disclosure provides processes including catalytic dehydrogenation cross-metathesis hydrogenation by: i) dehydrogenating C₃-C₅₀ cyclic alkanes and C₂-C₅₀ acyclic alkanes in a heavy naphtha range (e.g., coker naphtha; catalytic naphtha), including paraffins and naphthenes, to form C₂-C₅₀ acyclic olefin(s) and C₃-C₅₀ cyclic olefin(s); ii) treating the acyclic olefin(s) and the cyclic olefin(s) under conditions (e.g., ring-opening cross-metathesis, such as Ring Opening Metathesis Polymerization, “ROMP”) to form C₅-C₂₀₀ olefins, such as C₅-C₁₀₀ olefins, thus forming higher molecular weight compounds in the distillate range (or greater); iii) hydrogenating the C₅-C₂₀₀ olefins (such as C₅-C₂₀₀ polyolefin products), such as the C₅-C₁₀₀ olefins (such as C₅-C₁₀₀ polyolefin products, such as C₅-C₁₀₀ diolefin products), to form saturated products in the distillate range (or greater).

Processes of the present disclosure may be substantially thermo-neutral, meaning that the enthalpies of the products and reactants are similar, such that the reaction is not significantly endothermic, which would require higher temperatures. Thermo-neutral processes of the present disclosure can enable low temperature molecular weight growth of alkanes with low/reduced energy intensity (or greenhouse gas emission). Processes of the present disclosure may provide: i) little to no introduction of additional branches to the product(s) other than those carried from the feed; ii) little to no formation of light products if the feed composition is properly controlled; and/or iii) a wide range of operating temperatures (e.g., from about 0° C. to about 400° C.) for the ring-opening cross-metathesis process, allowing tailoring or reaction conditions (depending on feed and catalyst) to match the conditions for other further reactions (e.g., hydroisomerization, cyclization, aromatization, Diels-Alder, and/or alkylation).

For the purposes of the present disclosure, the numbering scheme for the Periodic Table Groups is used as described in CHEMICAL AND ENGINEERING NEWS, 63(5), pg. 27 (1985). Therefore, a “group 4 metal” is an element from group 4 of the Periodic Table, e.g., Hf, Ti, or Zr.

As used herein, and unless otherwise specified, the term “Cₙ” means hydrocarbon(s) having n carbon atom(s) per molecule, wherein n is a positive integer. As used herein, and unless otherwise specified, the term “hydrocarbon” means a class of compounds containing hydrogen bound to carbon, and encompasses (i) saturated hydrocarbon compounds, (ii) unsaturated hydrocarbon compounds, and (iii) mixtures of hydrocarbon compounds (saturated and/or unsaturated), including mixtures of hydrocarbon compounds having different values of n. Additionally, the hydrocarbon compound may contain, for example, heteroatoms such as sulphur, oxygen, nitrogen, or any combination thereof.

The term “acyclic alkanes” includes linear and branched acyclic alkanes, unless otherwise specified.

The term “acyclic alkenes” includes linear and branched acyclic alkenes, unless otherwise specified.

A “polymer” has two or more of the same or different monomer (“mer”) units. A “homopolymer” is a polymer having mer units that are the same. A “copolymer” is a polymer having two or more mer units that are different from each other. A “terpolymer” is a polymer having three mer units that are different from each other. “Different” as used to refer to mer units indicates that the mer units differ from each other by at least one atom or are different isomerically. Accordingly, the definition of copolymer, as used herein, includes terpolymers.

As used herein, the term “base stock” means a hydrocarbon liquid useable as a major component of a lubricating oil. As used herein, the term “base oil” refers to a blend of base stocks useable as a major component of a lubricating oil. As used herein, the term “major component” means a component present in a lubricating oil in an amount of about 50 weight percent (wt %) or greater. As used herein, the term “minor component” means a component (e.g., one or more lubricating oil additives) present in a lubricating oil in an amount less than about 50 wt %.

A “catalyst system” includes at least one catalyst compound and optionally, one or more activator(s). When “catalyst system” is used to describe the catalyst compound/activator combination before activation, it means the unactivated catalyst complex (precatalyst) together with an activator. When it is used to describe the combination after activation, it means the activated complex and the activator. The catalyst compound may be neutral as in a precatalyst, or a charged species with a counter ion as in an activated catalyst system. An example of a suitable activator can be tetramethyl tin, Me₄Sn.

In the description herein, the catalyst may be described as a catalyst precursor, a pre-catalyst compound, catalyst compound or a transition metal compound, and these terms are used interchangeably. A polymerization catalyst system is a catalyst system that can polymerize monomers to polymer.

For purposes of this disclosure and claims thereto, the term “substituted” means that a hydrogen atom in the compound or group in question has been replaced with a group or atom other than hydrogen. The replacing group or atom is called a substituent. Substituents can be, e.g., a substituted or unsubstituted hydrocarbyl group, a heteroatom, and the like. For example, a “substituted hydrocarbyl” is a group made of carbon and hydrogen where at least one hydrogen therein is replaced by a non-hydrogen atom or group. A heteroatom can be nitrogen, sulfur, oxygen, halogen, etc.

The term “alkenyl” means a straight-chain, branched-chain, or cyclic hydrocarbon radical having one or more double bonds. These alkenyl radicals may be optionally substituted. Examples of suitable alkenyl radicals can include ethenyl, propenyl, allyl, 1,4-butadienyl cyclopropenyl, cyclobutenyl, cyclopentenyl, cyclohexenyl, cycloctenyl, and the like, including their substituted analogues.

The term “alkoxy” or “alkoxide” means an alkyl ether or aryl ether radical where the term alkyl is as defined above. Examples of suitable alkyl ether radicals can include methoxy, ethoxy, n-propoxy, iso-propoxy, n-butoxy, iso-butoxy, sec-butoxy, tert-butoxy, phenoxyl, and the like.

The term “aryl” or “aryl group” means a six carbon aromatic ring and the substituted variants thereof, such as phenyl, 2-methyl-phenyl, xylyl, 4-bromo-xylyl. Likewise, heteroaryl means an aryl group where a ring carbon atom (or two or three ring carbon atoms) has been replaced with a heteroatom, such as N, O, or S. As used herein, the term “aromatic” also refers to pseudoaromatic heterocycles which are heterocyclic substituents that have similar properties and structures (nearly planar) to aromatic heterocyclic ligands, but are not by definition aromatic; likewise the term aromatic also refers to substituted aromatics.

Reference to an alkyl, alkenyl, alkoxide, or aryl group without specifying a particular isomer (e.g., butyl) expressly discloses all isomers (e.g., n-butyl, iso-butyl, sec-butyl, and tert-butyl).

For purposes of the present disclosure, “alkoxides” include those where the alkyl group is a C₁ to C₁₀ hydrocarbyl. The alkyl group may be straight chain, branched, or cyclic. The alkyl group may be saturated or unsaturated. In at least one embodiment, the alkyl group may include at least one aromatic group.

The terms “hydrocarbyl radical,” “hydrocarbyl,” and “hydrocarbyl group,” are used interchangeably. Likewise, the terms “group,” “radical,” and “ substituent” are also used interchangeably. For purposes of this disclosure, “hydrocarbyl radical” is defined to be C₁-C₁₀₀ radicals, that may be linear, branched, or cyclic, and when cyclic, aromatic or non-aromatic. Examples of such radicals can include methyl, ethyl, n-propyl, isopropyl, n-butyl, isobutyl, sec-butyl, tert-butyl, pentyl, iso-amyl, hexyl, octyl, cyclopropyl, cyclobutyl, cyclopentyl, cyclohexyl, cyclooctyl, and the like including their substituted analogues.

The term “aralkyl” means a univalent radical derived from an alkyl radical by replacing one or more hydrogen atoms by one or more aryl groups.

The term “alkaryl” means an aryl-substituted alkyl radical (e.g., propyl-phenyl), such as a radical in which an aryl group is substituted for a hydrogen atom of an alkyl group.

The term “alkynyl” (also referred to as “ynyl”) means a univalent aliphatic hydrocarbon radical derived from an alkyne.

The term “ring atom” means an atom that is part of a cyclic ring structure. By this definition, a benzyl group has six ring atoms and tetrahydrofuran has 5 ring atoms.

A heterocyclic ring is a ring having a heteroatom in the ring structure as opposed to a heteroatom substituted ring where a hydrogen on a ring atom is replaced with a heteroatom. For example, tetrahydrofuran is a heterocyclic ring and 4-N,N-dimethylamino-phenyl is a heteroatom-substituted ring.

The term “tandem reaction”, also referred to as “cascade reaction”, or “domino reaction”, refers to a chemical process including at least two or more consecutive reactions such that each subsequent reaction occurs by means of the chemical functionality formed in the previous reaction. The isolation of the intermediates formed during a tandem reaction may not be required. The reaction conditions of a tandem reaction might not change among the consecutive processes of a cascade and new reagents might not be added after the initial process. A “one-pot” sequence consisting of a single catalytic transformation and a subsequent stoichiometric modification does not constitute a tandem catalysis, even though the substrate has undergone two distinct transformations.

The term “olefin” refers to an unsaturated hydrocarbon compound having a hydrocarbon chain containing at least one carbon-to-carbon double bond in the structure thereof, wherein the carbon-to-carbon double bond does not constitute a part of an aromatic ring. The olefin may be linear, branched linear, or cyclic.

The term “terminal olefin” refers to an olefin having a terminal carbon-to-carbon double bond in the structure thereof ((R¹)(R²)C═CH₂, where R¹ and R² can be independently hydrogen or a hydrocarbyl group, such as R¹ is hydrogen, and R² is an alkyl group). A “linear terminal olefin” is a terminal olefin defined in this paragraph wherein R¹ is hydrogen, and R² is hydrogen or a linear alkyl group.

The term “vinyl” means an olefin having the following formula:

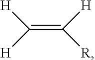

wherein R is a hydrocarbyl group, such as a saturated hydrocarbyl group.

The term “vinylidene” means an olefin having the following formula:

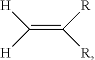

wherein each instance of R is independently a hydrocarbyl group, such as a saturated hydrocarbyl group.

The term “vinylene” or “1,2-di-substituted vinylene” means

(i) an olefin having the following formula (which is a “cis-” conformation):

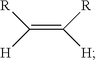

or
(ii) an olefin having the following formula (which is a “trans-” conformation):

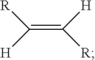

or
(iii) a mixture of (i) and (ii) at any proportion thereof,
wherein each instance of R is independently a hydrocarbyl group, such as saturated hydrocarbyl group.

The term “internal olefin” includes olefins that are vinylenes.

The term “tri-substituted vinylene” means an olefin having the following formula:

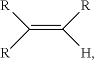

wherein each instance of R is independently a hydrocarbyl group, such as a saturated hydrocarbyl group.

The term “tetra-substituted vinylene” means an olefin having the following formula:

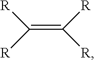

wherein each instance of R is independently a hydrocarbyl group, such as a saturated hydrocarbyl group.

An internal olefin (e.g., monomers) of the present disclosure can be a linear or branched C₄-C₅₀ olefin having one or more carbon-carbon double bonds along the olefin backbone (also referred to as “internal unsaturation”) instead of, or in addition to, a carbon-carbon double bond at a terminus of the olefin (also referred to as “terminal unsaturation”). Linear or branched C₄-C₅₀ internal olefins may be referred to as C₄-C₅₀ internal-olefins. In addition to internal unsaturations, a C₄-C₅₀ internal olefin may additionally have one or more terminal unsaturations. An internal olefin can have one or more cis-conformations or one or more trans-conformations.

In at least one embodiment, an internal olefin is selected from a cis-configuration, trans-configuration, or mixture thereof of one or more of 2-butene, 2-pentene, 2-hexene, 3-hexene, 2-heptene, 3-heptene, 2-octene, 3-octene, 4-octene, 2-nonene, 3-nonene, 4-nonene, 2-decene, 3-decene, 4-decene, and 5-decene. Internal olefins of the present disclosure can be obtained from commercial sources (such as Sigma Aldrich or TCI) and/or may be obtained from refined hydrocarbon feeds such as fluid catalytic cracking (FCC) gasoline or coker naphtha.

### Dehydrogenation Processes

The present disclosure provides processes for converting a hydrocarbon feedstock (e.g., heavy naphtha; biomass) comprising contacting the feedstock with a first catalyst, such as a dehydrogenation catalyst. The hydrocarbon feedstock to be dehydrogenated may include, in whole or in part, a liquefied petroleum gas (LPG), a naphtha stream, having a boiling point in the range of about 70° C. to about 185° C., a gas oil (e.g., light, medium, or heavy gas oil) having an initial boiling point above 200° C., a 50% point of at least 260° C. and an end point of at least 350° C. The feedstock may also include vacuum gas oils, thermal oils, residual oils, cycle stocks, whole top crudes, tar sand oils, shale oils, synthetic fuels, heavy hydrocarbon fractions derived from the destructive hydrogenation of coal, tar, pitches, asphalts, hydrotreated feedstocks derived from any of the foregoing.

Heavy naphtha includes both paraffins and naphthenes (e.g., derived from coal, shale, or petroleum). For example, a naphtha may include from about 15 wt % to about 30 wt % paraffins, from about 5 wt % to about 20 w t% cyclo-paraffins, from about 10 wt % to about 30 wt % olefins, from about 1 wt % to about 10 wt % cycloolefins, and from about 10 wt % to about 40 wt % aromatics. Heavy naphtha can be converted to olefins, such as mono-olefins, using dehydrogenation. The heavy naphtha feed can be processed “as-is”, or optionally separated into paraffin and naphthene fractions, or further fractionated to individual carbon number. The naphtha feed may include one or more of n-hexane, n-heptane, cyclohexane, methylcyclohexane, methylcyclopentane, benzene, toluene, xylenes, or a mixture thereof. Dehydrogenation processes of the present disclosure include the dehydrogenation of C₂-C₅₀ acyclic alkanes and C₃-C₅₀ cyclic alkanes in a heavy naphtha range (e.g., coker naphtha;

catalytic naphtha), including paraffins and/or naphthenes, to form C₂-C₅₀ acyclic olefins and C₃-C₅₀ cyclic olefins.

Furthermore, the feed composition can be controlled by introducing, injecting, feeding, co-feeding, a defined amount of a defined hydrocarbon starting materials, thus by controlling the ratio of the starting material. Accordingly, the average molecular weight of the products can be controlled subsequently. For instance, longer molecular weight range products can be produced when less acyclic starting material (e.g., linear acyclic paraffins) is introduced to the feed. For example, when the hydrocarbon starting material is one or more Cₙ cyclic alkane(s), with n being the number of carbons of the alkane, and x being the number of cyclic alkane used for the reaction, the average molecular weight of the product can be defined as [xCₙ+carbon number of the acyclic alkane feed], with x≥2, and/or 3≤n≤100). For example, an average molecular weight of the product formed via combination of three cyclopentane molecules and one propane molecule can be [3C₅+C₃=C₁₈].

Alternatively, shorter molecular weight range products (e.g., shorter range diesel) can be produced when more linear acyclic starting material is added to the feed. For example, linear acyclic starting material, such as linear acyclic paraffins, can be combined with one or more cyclic and/or acyclic alkane(s) either before, or after, alternatively before and after, the introduction of one or more cyclic and acyclic alkane(s) into the reactor.

A dehydrogenation process can involve contacting a C₃-C₅₀ cyclic alkane and a C₂-C₅₀ acyclic alkane feed with a catalyst system including platinum group metals, alloys, oxides, carbides, nitrides, and/or sulfides of individual transition metal and/or a mixed metal catalyst. The catalyst system can be bulk and/or supported. Suitable supports are non-acidic oxides including silica, aluminas, zirconia, titania, ceria, non-acidic clays, or basic oxides (such as magnesia, hydrotalcites, or lanthanum oxide). The catalyst system may include a transition metal oxide, such as CuO, Ag₂O, ZnO, NiO, CoOₓ, FeOₓ, MnOₓ, CrOₓ, or VOₓ, for example, or mixtures thereof, where x can be 1 to 3.5. In at least one embodiment, the dehydrogenation process is mediated by copper oxide (CuO). C₂-C₅₀ acyclic olefins and C₃-C₅₀ cyclic olefins products can be substituted and/or non-substituted olefins products.

In a dehydrogenation process, a feed stream including at least 2 wt % of C₂ to C₅₀ cyclic alkanes and C₂ to C₅₀ acyclic alkanes can be contacted with a catalyst suitable for a dehydrogenation process, with or without the presence of a solvent, such as the hydrocarbons including C₃ to C₅₀ cyclic alkanes and C₂ to C₅₀ acyclic alkanes of the feed stream can be used directly as solvent.

Optionally one or more solvent(s) can be used for the process of the present disclosure. The solvent may be a saturated hydrocarbon or an aromatic solvent such as n-hexane, n-heptane, cyclohexane, benzene, toluene, xylenes, or a mixture thereof. Contacting the catalyst with a feedstream comprising the C₂ to C₅₀ alkanes may be carried out in an atmosphere inert under the process conditions, such as in nitrogen, argon, or a mixture thereof. Naphtha, including both paraffins and naphthenes, may include various ranges of cyclic and acyclic alkanes. Hence, controlling a co-feed molar ratio of cyclic alkanes to acyclic alkanes starting materials provides control of the molecular weight of the C₅-C₂₀₀ olefin products, such as the C₅-C₁₀₀ olefin products. For example, C₃-C₅₀ cyclic alkanes can be cyclopropane, cyclobutane, cyclopentane, cyclohexane, cycloheptane, cyclooctane. Examples of C₂-C₅₀ acyclic alkanes can be ethane, propane, butane, pentane, hexane, heptane, octane.

A molar ratio of one or more cyclic alkanes to acyclic alkanes can be from about 1:1000 to about 1000:1, such as from about 1:700 to about 700:1, such as from about 1:500 to about 500:1, such as from about 1:250 to about 250:1, such as from about 1:100 to about 100:1, such as from about 1:50 to about 50:1, such as from about 1:10 to about 10:1.

In at least one embodiment, a dehydrogenation process is performed at a temperature of 450° C. or less, such as from about 100° C. to about 450° C., such as from about 150° C. to about 350° C. (e.g., 275° C.). A dehydrogenation process of the present disclosure may be carried out by mixing a solution of C₃-C₅₀ cyclic alkanes and C₂-C₅₀ acyclic alkanes and the catalyst(s), cooling the solution, and optionally allowing the mixture to increase in temperature. A dehydrogenation process can be performed at a pressure greater than 1 bar gauge, such as from about 1 bar gauge to about 2,000 bar gauge, such as about 1 bar gauge to about 1,000 bar gauge, such as about 1 bar gauge to about 750 bar gauge, and/or for a period of time of from about 5 minutes to about 20 hours, such as from about 30 minutes to about 4 hours, such as from about 1 hour to about 3 hours. In at least one embodiment, dehydrogenation is performed at a temperature higher than 400° C.; and/or at a pressure of from about less than 1 bar gauge to about 2 bar gauge.

In at least one embodiment, the process for the production of a C₂-C₅₀ acyclic olefin of Formula (I) and a C₃-C₅₀ cyclic olefin of Formula (II) includes: dehydrogenating at least one C₂-C₅₀ acyclic alkane and at least one C₃-C₅₀ cyclic alkane by contacting the at least one C₂-C₅₀ acyclic alkane and the at least one C₃-C₅₀ cyclic alkane with a catalyst system in at least one solution dehydrogenation reactor at a reactor pressure of from 1 bar gauge to 2000 bar gauge and a reactor temperature of from about 100° C. to about 450° C. The C₂-C₅₀ acyclic olefins (I) and C₃-C₅₀ cyclic olefins (II) products can be recovered and analyzed by GC.

### Dehydrogenation Catalysts

Dehydrogenation catalyst(s) of the present disclosure may include platinum group metals (e.g., Pd, Rh, Pt), alloys (e.g., bimetallic Pt—Fe catalysts, Cu—Al alloy catalyst, Pt—Zn alloy nanocluster catalyst), oxides, carbides (e.g., bulk W—Mo mixed carbides, Mo carbide modified nanocarbon catalysts), nitrides (e.g., B—N catalyst), and/or sulfides (e.g., Mo-sulfide-alumina catalyst) of individual transition metal and/or mixed metal catalyst. The catalyst system can be bulk and/or supported. The catalyst system may include a transition metal oxide, such as copper oxide (CuO), silver oxide (Ag₂O), zinc oxide (ZnO), nickel oxide (NiO), chromium oxide (CrOₓ), or vanadium oxide (VOₓ), CoOₓ, FeOₓ, MnOₓ, for example, or mixtures thereof, where x is in the range of 1 to 3.5. In at least one embodiment, the dehydrogenation process of the alkanes is mediated by CuO.

For purposes of the present disclosure, a catalyst loading % (based on the concentration of the alkanes) can be from about 0.01 mol % to about 50 mol %, such as from about 0.1 mol % to about 25 mol %, such as from about 0.2 mol % to about 10 mol %, such as from about 0.5 mol % to about 5 mol %, such as about 0.2 mol %, for example.

Optional Support Materials for Dehydrogenation Catalysts, Metathesis Catalysts, and/or Hydrogenation Catalysts

In embodiments herein, the catalyst system may include an inert support material. The supported material can be a porous support material, for example, talc, and inorganic oxides. Suitable supports are non-acidic oxides including silica, theta-alumina or any suitable aluminas, zirconia, titania, ceria, non-acidic clays, or basic oxides (such as magnesia, hydrotalcites, or lanthanum oxide). Other support materials may include zeolites, organoclays, or another organic or inorganic support material, or mixtures thereof.

The support material can be an inorganic oxide in a finely divided form. Suitable inorganic oxide materials for use in catalyst systems herein include groups 2, 4, 10, 11, 12, 13, and 14 metal oxides, such as silica, alumina, MgO, TiO₂, ZrO₂, rare-earth oxides (e.g., La₂O₃, CeO₂), and mixtures thereof. Other inorganic oxides that may be employed either alone or in combination with the silica, or alumina, are magnesia, titania, zirconia. Suitable supports may include magnesia, titania, zirconia, montmorillonite, phyllosilicate, zeolites, talc, clays. Also, combinations of these support materials may be used, for example, silica-chromium, silica-alumina, silica-titania. Support materials include Al₂O₃, ZrO₂, SiO₂, and combinations thereof, such as SiO₂, Al₂O₃, or SiO₂/Al₂O₃.

The support material should be dry, that is, free of absorbed water. Drying of the support material can be effected by heating or calcining at about 100° C. to about 1000° C., such as at least about 400° C. When the support material is silica, it is heated to at least 110° C., such as from about 110° C. to about 850° C., such as at about 600° C., for example; and/or for a time of about 1 minute to about 100 hours, from about 12 hours to about 72 hours, or from about 24 hours to about 60 hours. The calcined support material must have at least some reactive hydroxyl (OH) groups to produce supported catalyst systems of the present disclosure. The calcined support material is then contacted with at least one dehydrogenation/ring-opening metathesis/hydrogenation catalyst system comprising at least one dehydrogenation catalyst compound, at least one ring-opening metathesis catalyst compound, and/or at least one hydrogenation catalyst compound.

The support material, having reactive surface groups, such as hydroxyl groups, can be slurried in a non-polar solvent and the resulting slurry can be contacted with a solution of a catalyst compound(s). In at least one embodiment, the slurry of the support material is first contacted with a first catalyst compound, such as a dehydrogenation catalyst compound for a period of time in the range of from about 0.5 hours to about 24 hours, from about 2 hours to about 16 hours, or from about 4 hours to about 8 hours. Then, a solution of a second catalyst compound, such as a ring-opening metathesis catalyst compound, can be contacted with the isolated support/first catalyst compound. In at least one embodiment, the supported catalyst system is generated in situ. In alternate embodiment, the slurry of the support material is first contacted with the first catalyst compound for a period of time in the range of from about 0.5 hours to about 24 hours, from about 2 hours to about 16 hours, or from about 4 hours to about 8 hours. The slurry of the supported catalyst compound is then contacted with the second catalyst compound solution. Then a third catalyst compound (e.g., hydrogenation catalyst) can be added, as a solution or neat, to the solution mixture including the first and the second catalysts.

The mixture of the catalyst compounds and support can be heated to about 0° C. to about 70° C., such as about 23° C. to about 60° C., such as at room temperature. Contact times may range from about 0.5 hours to about 24 hours, from about 2 hours to about 16 hours, or from about 4 hours to about 8 hours.

Suitable non-polar solvents can be materials in which all of the reactants used herein, e.g., the first catalyst compound and the second catalyst compound are at least partially soluble and which are liquid at reaction temperatures. Non-polar solvents can be alkanes, such as isopentane, hexane, n-heptane, octane, nonane, and decane, although a variety of other materials including cycloalkanes, such as cyclohexane, aromatics, such as benzene, toluene, and ethylbenzene, may also be employed.

### Dehydrogenation Products

The present disclosure relates to compositions of matter produced by the methods described herein.

In at least one embodiment, a process described herein produces C₂-C₅₀ acyclic olefins of Formula (I) (such as ethene, propene, butene, pentene, hexene, heptene, octene, etc., and any isomers thereof), and C₃-C₅₀ cyclic olefins of Formula (II) (such as cyclopentene, methyl-cyclopentene, cyclohexene, cycloheptene, cyclooctene, norbornene, etc., and any isomers thereof).

In at least one embodiment, an acyclic olefin monomer is represented by formula (I):

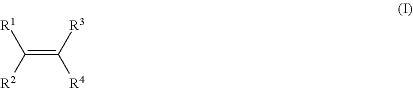

wherein:
R¹, R², R³, and R⁴ are independently hydrogen, C₁-C₄₀ hydrocarbyl (e.g., C₁-C₂₀ alkyl, C₂-C₂₀ alkenyl, C₂-C₂₀ alkynyl, C₅-C₂₀ aryl, C₆-C₂₄ alkaryl, C₆-C₂₄ aralkyl, C₁-C₂₀ alkyl, C₅-C₂₀ aryl, C₅-C₃₀ aralkyl, or C₅-C₃₀ alkaryl), C₁-C₄₀ substituted hydrocarbyl (e.g., substituted C₁-C₂₀ alkyl, C₂-C₂₀ alkenyl, C₂-C₂₀ alkynyl, C₅-C₂₀ aryl, C₆-C₂₄ alkaryl, C₆-C₂₄ aralkyl, C₁-C₂₀ alkyl, C₅-C₂₀ aryl, C₅-C₃₀ aralkyl, or C₅-C₃₀ alkaryl), a heteroatom or a heteroatom-containing group, such as each of R¹, R², R³, and R⁴ is independently selected from hydrogen, methyl, ethyl, propyl, butyl, pentyl, hexyl, heptyl, octyl, nonyl, decyl, undecyl, dodecyl, phenyl, substituted phenyl, biphenyl or an isomer thereof, which may be halogenated (such as bromopropyl, bromopropyl, bromobutyl, (bromomethyl)cyclopropyl, chloroethyl, 2,3,5,6-tetrafluorobenzyl, perfluoropropyl, perfluorobutyl, perfluoroethyl, perfluoromethyl), substituted hydrocarbyl radicals and isomers of substituted hydrocarbyl radicals such as trimethylsilylpropyl, trimethylsilylmethyl, trimethylsilylethyl, phenyl, or isomers of hydrocarbyl substituted phenyl such as methylphenyl, dimethylphenyl, trimethylphenyl, tetramethylphenyl, pentamethylphenyl, diethylphenyl, triethylphenyl, propylphenyl, dipropylphenyl, tripropylphenyl, dimethylethylphenyl, dimethylpropylphenyl, dimethylbutylphenyl, and dipropylmethylphenyle.

In at least one embodiment, R² and R³ are independently hydrogen or C₁-C₄₀ hydrocarbyl (e.g., C₁-C₂₀ alkyl, C₂-C₂₀ alkenyl, C₂-C₂₀ alkynyl, C₅-C₂₀ aryl, C₆-C₂₄ alkaryl, C₆-C₂₄ aralkyl, C₁-C₂₀ alkyl, C₅-C₂₀ aryl, C₅-C₃₀ aralkyl, or C₅-C₃₀ alkaryl), substituted hydrocarbyl (e.g., substituted C₁-C₂₀ alkyl, C₂-C₂₀ alkenyl, C₂-C₂₀ alkynyl, C₅-C₂₀ aryl, C₆-C₂₄ alkaryl, C₆-C₂₄ aralkyl, C₁-C₂₀ alkyl, C₅-C₂₀ aryl, C₅-C₃₀ aralkyl, or C₅-C₃₀ alkaryl), such as methyl, ethyl, propyl, butyl, pentyl, hexyl, heptyl, octyl, nonyl, decyl, undecyl, dodecyl, phenyl, substituted phenyl, biphenyl or an isomer thereof, such as perfluoropropyl-, perfluorobutyl-, perfluoroethyl-, or perfluoromethyl-substituted hydrocarbyl radicals and isomers of substituted hydrocarbyl radicals such as trimethylsilylpropyl, trimethylsilylmethyl, trimethylsilylethyl, or phenyl, and isomers of hydrocarbyl substituted phenyl such as methylphenyl, dimethylphenyl, trimethylphenyl, tetramethylphenyl, pentamethylphenyl, diethylphenyl, triethylphenyl, propylphenyl, dipropylphenyl, tripropylphenyl, dimethylethylphenyl, dimethylpropylphenyl, dimethylbutylphenyl, and dipropylmethylphenyl; and R¹ and R⁴ are independently selected from hydrogen, methyl, ethyl, propyl, butyl, pentyl, hexyl, heptyl, octyl, nonyl, decyl, undecyl, dodecyl, phenyl, substituted phenyl, biphenyl or an isomer thereof, which may be halogenated (such as bromopropyl, bromopropyl, bromobutyl, (bromomethyl)cyclopropyl, chloroethyl, 2,3,5,6-tetrafluorobenzyl, perfluoropropyl, perfluorobutyl, perfluoroethyl, perfluoromethyl), substituted hydrocarbyl radicals and isomers of substituted hydrocarbyl radicals such as trimethylsilylpropyl, trimethylsilylmethyl, trimethylsilylethyl, phenyl, or isomers of hydrocarbyl substituted phenyl such as methylphenyl, dimethylphenyl, trimethylphenyl, tetramethylphenyl, pentamethylphenyl, diethylphenyl, triethylphenyl, propylphenyl, dipropylphenyl, tripropylphenyl, dimethylethylphenyl, dimethylpropylphenyl, dimethylbutylphenyl, and dipropylmethylphenyl.

In at least one embodiment, R² and R³ are hydrogen and R¹ and R⁴ are independently selected from hydrogen, methyl, ethyl, propyl, butyl, pentyl, hexyl, heptyl, octyl, nonyl, decyl, undecyl, dodecyl, phenyl, substituted phenyl, biphenyl or an isomer thereof, which may include oxygen, nitrogen, and/or sulfur (such as methoxypropyl, methoxybutyl, methoxypentyl methoxyhexyl, methoxyheptyl, methoxyoctyl, methoxydodecyl, ethoxyethyl, ethoxypropyl, ethoxybutyl, ethoxypentyl ethoxyhexyl, ethoxyheptyl, ethoxyoctyl, ethoxyldecyl, ethoxydodecyl, ethoxyphenyl, 1-aminoalkyl (e.g., 1-aminobutyl), 2-aminoalkyl (e.g., 2-aminopentyl), 1-alkylaminoalkyl (e.g., 1-methylaminopropyl), dialkylaminoalkyl (e.g., dimethylaminoethyl) or isomers of hydrocarbyl substituted phenyl such as methylphenyl, dimethylphenyl, trimethylphenyl, tetramethylphenyl, pentamethylphenyl, diethylphenyl, triethylphenyl, propylphenyl, dipropylphenyl, tripropylphenyl, dimethylethylphenyl, dimethylpropylphenyl, dimethylbutylphenyl, and dipropylmethylphenyl.

For example, the acyclic olefin monomer represented by formula (I) can be a vinylenes, such as an olefin with a “cis-” conformation, such as an olefin with “trans-” conformation, or a mixture thereof, thus at any proportion thereof. Furthermore, the acyclic olefin monomer can be a tri-substituted vinylene. Traces of tetra-substituted vinylene may be present in the reaction mixture.

In at least one embodiment, a cyclic olefin compound is represented by formula (II):

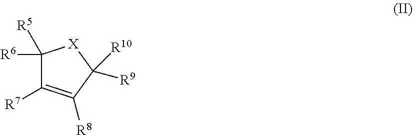

wherein:
X is a one-atom to five-atom linkage (with a “one-atom” linkage referring to a linkage that provides a single, optionally substituted atom between the two adjacent carbon atoms, and a “five-atom” linkage, similarly, referring to a linkage that provides five optionally substituted atoms between the two adjacent carbon atoms); In at least one embodiment, and when the monomer is bicyclic (e.g., when R⁵ and R¹⁰ are linked), then X is a one-atom or two-atom linkage, such as a linkage that has one or two optionally substituted atoms between the two carbon atoms to which X is bound. For example, X can be of the formula —CR¹¹R¹²—(X¹)_{q}—wherein q is zero or 1, X¹ is CR¹³R¹⁴, O, S, or NR¹⁵, and R¹¹, R¹², R¹³, R¹⁴, and R¹⁵ are independently selected from hydrogen, hydrocarbyl (e.g., C₁-C₂₀ alkyl, C₂-C₂₀ alkenyl, C₂-C₂₀ alkynyl, C₅-C₂₀ aryl, C₆-C₂₄ alkaryl, C₆-C₂₄ aralkyl, C₁-C₂₀ alkyl, C₅-C₂₀ aryl, C₅-C₃₀ aralkyl, or C₅-C₃₀ alkaryl), substituted hydrocarbyl (e.g., substituted C₁-C₂₀ alkyl, C₂-C₂₀ alkenyl, C₂-C₂₀ alkynyl, C₅-C₂₀ aryl, C₆-C₂₄ alkaryl, C₆-C₂₄ aralkyl, C₁-C₂₀ alkyl, C₅-C₂₀ aryl, C₅-C₃₀ aralkyl, or C₅-C₃₀ alkaryl), heteroatom-containing hydrocarbyl (e.g., C₁-C₂₀ heteroalkyl, C₅-C₂₀ heteroaryl, heteroatom-containing C₅-C₃₀ aralkyl, or heteroatom-containing C₅-C₃₀ alkaryl), substituted heteroatom-containing hydrocarbyl (e.g., substituted C₁-C₂₀ heteroalkyl, C₅-C₂₀ heteroaryl, heteroatom-containing C₅-C₃₀ aralkyl, or heteroatom-containing C₅-C₃₀ alkaryl); When q is 1, suitable examples of linkages can be wherein X¹ is CR¹³R¹⁴, thus providing a substituted or unsubstituted ethylene moiety to the cyclic olefin of Formula (IV). Accordingly, when R¹¹, R¹², R¹³, and R¹⁴ are hydrogen, then X is ethylene. When q is zero, the linkage can be substituted or unsubstituted methylene, and a suitable linkage within this group can be methylene (e.g., when R¹¹ and R¹² are both hydrogen);
At least one of R⁷ and R⁸ is hydrogen and the other is selected from hydrogen, hydrocarbyl (e.g., C₁-C₂₀ alkyl, C₂-C₂₀ alkenyl, C₂-C₂₀ alkynyl, C₅-C₂₀ aryl, C₆-C₂₄ alkaryl, and C₆-C₂₄ aralkyl), substituted hydrocarbyl (e.g., substituted C₁-C₂₀ alkyl, C₂-C₂₀ alkenyl, C₂-C₂₀ alkynyl, C₅-C₂₀ aryl, C₆-C₂₄ alkaryl, and C₆-C₂₄ aralkyl), heteroatom-containing hydrocarbyl (e.g., heteroatom-containing C₁-C₂₀ alkyl, C₂-C₂₀ alkenyl, C₂-C₂₀ alkynyl, C₅-C₂₀ aryl, C₆-C₂₄ alkaryl, and C₆-C₂₄ aralkyl), or substituted heteroatom-containing hydrocarbyl (e.g., substituted heteroatom-containing C₁-C₂₀ alkyl, C₂-C₂₀ alkenyl, C₂-C₂₀ alkynyl, C₅-C₂₀ aryl, C₆-C₂₄ alkaryl, and C₆-C₂₄ aralkyl); and
R⁵, R⁶, R⁹, and R¹⁰ are independently selected from hydrogen, hydrocarbyl (e.g., C₁-C₂₀ alkyl, C₅-C₂₀ aryl, C₅-C₃₀ aralkyl, or C₅-C₃₀ alkaryl), substituted hydrocarbyl (e.g., substituted C₁-C₂₀ alkyl, C₅-C₂₀ aryl, C₅-C₃₀ aralkyl, or C₅-C₃₀ alkaryl), heteroatom-containing hydrocarbyl (e.g., C₁-C₂₀ heteroalkyl, C₅-C₂₀ heteroaryl, heteroatom-containing C₅-C₃₀ aralkyl, or heteroatom-containing C₅-C₃₀ alkaryl), substituted heteroatom-containing hydrocarbyl (e.g., substituted C₁-C₂₀ heteroalkyl, C₅-C₂₀ heteroaryl, heteroatom-containing C₅-C₃₀ aralkyl, or heteroatom-containing C₅-C₃₀ alkaryl). Additionally, any two or more of R⁵, R⁶, R⁹, and R¹⁰ can be taken together to form a cyclic group, which may be, for example, five- or six-membered rings, or two or three five- or six-membered rings, which may be either fused or linked. The cyclic groups may be aliphatic or aromatic, and may be heteroatom-containing and/or substituted.

One group of such cyclic olefins are those of formula (II) wherein R⁶ and R¹⁰ are hydrogen, R⁵ is and R⁹ combine to form a cyclic ring. In such embodiments, the cyclic olefin is represented by Formula (IV):

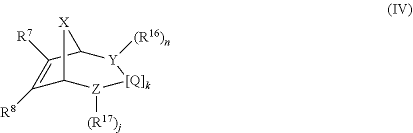

wherein:
X is a one-atom to five-atom linkage. In at least one embodiment, and when the monomer is bicyclic (e.g., when R⁵ and R¹⁰ are linked in Formula (II)), then X is a one-atom or two-atom linkage, such as a linkage that has one or two optionally substituted atoms between the two carbon atoms to which Xis bound. For example, X can be of the formula —CR¹¹R¹²—(X¹)_{q}— wherein q is zero or 1, X¹ is CR¹³R¹⁴, O, S, or NR¹⁵, and R¹¹, R¹², R¹³, R¹⁴, and R¹⁵ are independently selected from hydrogen, hydrocarbyl (e.g., C₁-C₂₀ alkyl, C₂-C₂₀ alkenyl, C₂-C₂₀ alkynyl, C₅-C₂₀ aryl, C₆-C₂₄ alkaryl, C₆-C₂₄ aralkyl, C₁-C₂₀ alkyl, C₅-C₂₀ aryl, C₅-C₃₀ aralkyl, or C₅-C₃₀ alkaryl), substituted hydrocarbyl (e.g., substituted C₁-C₂₀ alkyl, C₂-C₂₀ alkenyl, C₂-C₂₀ alkynyl, C₅-C₂₀ aryl, C₆-C₂₄ alkaryl, C₆-C₂₄ aralkyl, C₁-C₂₀ alkyl, C₅-C₂₀ aryl, C₅-C₃₀ aralkyl, or C₅-C₃₀ alkaryl), heteroatom-containing hydrocarbyl (e.g., C₁-C₂₀ heteroalkyl, C₅-C₂₀ heteroaryl, heteroatom-containing C₅-C₃₀ aralkyl, or heteroatom-containing C₅-C₃₀ alkaryl), substituted heteroatom-containing hydrocarbyl (e.g., substituted C₁-C₂₀ heteroalkyl, C₅-C₂₀ heteroaryl, heteroatom-containing C₅-C₃₀ aralkyl, or heteroatom-containing C₅-C₃₀ alkaryl); When q is 1, suitable examples of linkages can be wherein X¹ is CR¹³R¹⁴, thus providing a substituted or unsubstituted ethylene moiety to the cyclic olefin of Formula (IV). Accordingly, when R¹¹, R¹², R¹³, and R¹⁴ are hydrogen, then Xis ethylene. When q is zero, the linkage can be substituted or unsubstituted methylene, and a suitable linkage within this group can be methylene (e.g., when R¹¹ and R¹² are both hydrogen);
At least one of R⁷ and R⁸ is hydrogen and the other is selected from hydrogen, hydrocarbyl (e.g., C₁-C₂₀ alkyl, C₂-C₂₀ alkenyl, C₂-C₂₀ alkynyl, C₅-C₂₀ aryl, C₆-C₂₄ alkaryl, and C₆-C₂₄ aralkyl), substituted hydrocarbyl (e.g., substituted C₁-C₂₀ alkyl, C₂-C₂₀ alkenyl, C₂-C₂₀ alkynyl, C₅-C₂₀ aryl, C₆-C₂₄ alkaryl, and C₆-C₂₄ aralkyl), heteroatom-containing hydrocarbyl (e.g., heteroatom-containing C₁-C₂₀ alkyl, C₂-C₂₀ alkenyl, C₂-C₂₀ alkynyl, C₅-C₂₀ aryl, C₆-C₂₄ alkaryl, and C₆-C₂₄ aralkyl), or substituted heteroatom-containing hydrocarbyl (e.g., substituted heteroatom-containing C₁-C₂₀ alkyl, C₂-C₂₀ alkenyl, C₂-C₂₀ alkynyl, C₅-C₂₀ aryl, C₆-C₂₄ alkaryl, and C₆-C₂₄ aralkyl);
Y and Z are independently N, O, or S;
k is zero or 1;
j and n are independently zero or 1;
Q is a one-atom to five-atom linkage. In at least one embodiment, and when the monomer is bicyclic (e.g., when R¹⁶ and R¹⁷ are linked), then Q is a one-atom or two-atom linkage, such as a linkage that has one or two optionally substituted atoms between the two carbon atoms to which Q is bound. For example, Q can be of the formula —CR¹¹R¹²—(Q¹)_{q}′—wherein q′ is zero or 1, Q¹ is CR¹³R¹⁴′, O, S, or NR¹⁵′, and R¹¹, R¹²′, R¹³′, R¹⁴′, and R¹⁵′ are independently selected from hydrogen, hydrocarbyl (e.g., C₁-C₂₀ alkyl, C₂-C₂₀ alkenyl, C₂-C₂₀ alkynyl, C₅-C₂₀ aryl, C₆-C₂₄ alkaryl, C₆-C₂₄ aralkyl, C₁-C₂₀ alkyl, C₅-C₂₀ aryl, C₅-C₃₀ aralkyl, or C₅-C₃₀ alkaryl), substituted hydrocarbyl (e.g., substituted C₁-C₂₀ alkyl, C₂-C₂₀ alkenyl, C₂-C₂₀ alkynyl, C₅-C₂₀ aryl, C₆-C₂₄ alkaryl, C₆-C₂₄ aralkyl, C₁-C₂₀ alkyl, C₅-C₂₀ aryl, C₅-C₃₀ aralkyl, or C₅-C₃₀ alkaryl), heteroatom-containing hydrocarbyl (e.g., C₁-C₂₀ heteroalkyl, C₅-C₂₀ heteroaryl, heteroatom-containing C₅-C₃₀ aralkyl, or heteroatom-containing C₅-C₃₀ alkaryl), substituted heteroatom-containing hydrocarbyl (e.g., substituted C₁-C₂₀ heteroalkyl, C₅-C₂₀ heteroaryl, heteroatom-containing C₅-C₃₀ aralkyl, or heteroatom-containing C₅-C₃₀ alkaryl); When q′ is 1, suitable examples of linkages can be wherein Q¹ is CR¹³′R¹⁴′, thus providing a substituted or unsubstituted ethylene moiety to the cyclic olefin of Formula (IV). Accordingly, when R¹¹′, R¹²′, R¹³′, and R^{14A}′ are hydrogen, then Q is ethylene. When q′ is zero, the linkage can be substituted or unsubstituted methylene, and a suitable linkage within this group can be methylene (e.g., when R¹¹′ and R¹²′ are both hydrogen);
R¹⁶ and R¹⁷ are independently selected from hydrogen, hydrocarbyl, substituted hydrocarbyl, heteroatom-containing hydrocarbyl, substituted heteroatom-containing hydrocarbyl, and amino groups, wherein R¹⁶ and R¹⁷ may be taken together to form a cyclic group;
when Y is O or S, then n is zero;
when Z is O or S, then j is zero;
when Yis N, then n is 1; and
when Z is N, then j is 1.

In an alternate embodiment, R⁶ and R⁹ of formula (II) are hydrogen, in which case the cyclic olefin is represented by formula (V):

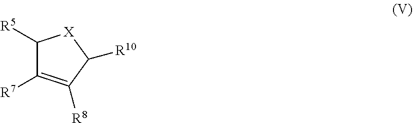

wherein:
X is a one-atom to five-atom linkage. In at least one embodiment, and when the monomer is bicyclic (e.g., when R⁵ and R¹⁰ are linked), then X is a one-atom or two-atom linkage, such as a linkage that has one or two optionally substituted atoms between the two carbon atoms to which Xis bound. For example, X can be of the formula —CR¹¹R¹²—(X¹)_{q}— wherein q is zero or 1, X¹ is CR¹³R¹⁴, O, S, or NR¹⁵, and R¹¹, R¹², R¹³, R¹⁴, and R¹⁵ are independently selected from hydrogen, hydrocarbyl (e.g., C₁-C₂₀ alkyl, C₂-C₂₀ alkenyl, C₂-C₂₀ alkynyl, C₅-C₂₀ aryl, C₆-C₂₄ alkaryl, C₆-C₂₄ aralkyl, C₁-C₂₀ alkyl, C₅-C₂₀ aryl, C₅-C₃₀ aralkyl, or C₅-C₃₀ alkaryl), substituted hydrocarbyl (e.g., substituted C₁-C₂₀ alkyl, C₂-C₂₀ alkenyl, C₂-C₂₀ alkynyl, C₅-C₂₀ aryl, C₆-C₂₄ alkaryl, C₆-C₂₄ aralkyl, C₁-C₂₀ alkyl, C₅-C₂₀ aryl, C₅-C₃₀ aralkyl, or C₅-C₃₀ alkaryl), heteroatom-containing hydrocarbyl (e.g., C₁-C₂₀ heteroalkyl, C₅-C₂₀ heteroaryl, heteroatom-containing C₅-C₃₀ aralkyl, or heteroatom-containing C₅-C₃₀ alkaryl), substituted heteroatom-containing hydrocarbyl (e.g., substituted C₁-C₂₀ heteroalkyl, C₅-C₂₀ heteroaryl, heteroatom-containing C₅-C₃₀ aralkyl, or heteroatom-containing C₅-C₃₀ alkaryl). When q is 1, suitable examples of linkages can be wherein X¹ is CR¹³R¹⁴, thus providing a substituted or unsubstituted ethylene moiety. Accordingly, when R¹¹, R¹², R¹³ and R¹⁴ are hydrogen, then X is ethylene. When q is zero, the linkage can be substituted or unsubstituted methylene, and a suitable linkage within this group can be methylene (e.g., when R¹¹ and R¹² are both hydrogen).

In at least one embodiment, one of R⁷ and R⁸ is hydrogen and the other is selected from hydrogen, hydrocarbyl (e.g., C₁-C₂₀ alkyl, C₂-C₂₀ alkenyl, C₂-C₂₀ alkynyl, C₅-C₂₀ aryl, C₆-C₂₄ alkaryl, and C₆-C₂₄ aralkyl), substituted hydrocarbyl (e.g., substituted C₁-C₂₀ alkyl, C₂-C₂₋₀ alkenyl, C₂-C₂₀ alkynyl, C₅-C₂₀ aryl, C₆-C₂₄ alkaryl, and C₆-C₂₄ aralkyl), heteroatom-containing hydrocarbyl (e.g., heteroatom-containing C₁-C₂₀ alkyl, C₂-C₂₀ alkenyl, C₂-C₂₀ alkynyl, C₅-C₂₀ aryl, C₆-C₂₄ alkaryl, and C₆-C₂₄ aralkyl), or substituted heteroatom-containing hydrocarbyl (e.g., substituted heteroatom-containing C₁-C₂₀ alkyl, C₂-C₂₀ alkenyl, C₂-C₂₀ alkynyl, C₅-C₂₀ aryl, C₆-C₂₄ alkaryl, and C₆-C₂₄ aralkyl).

In at least one embodiment, R⁵, R⁶, R⁹, and R¹⁰ are independently selected from hydrogen, hydrocarbyl (e.g., C₁-C₂₀ alkyl, C₅-C₂₀ aryl, C₅-C₃₀ aralkyl, or C₅-C₃₀ alkaryl), substituted hydrocarbyl (e.g., substituted C₁-C₂₀ alkyl, C₅-C₂₀ aryl, C₅-C₃₀ aralkyl, or C₅-C₃₀ alkaryl), heteroatom-containing hydrocarbyl (e.g., C₁-C₂₀ heteroalkyl, C₅-C₂₀ heteroaryl, heteroatom-containing C₅-C₃₀ aralkyl, or heteroatom-containing C₅-C₃₀ alkaryl), substituted heteroatom-containing hydrocarbyl (e.g., substituted C₁-C₂₀ heteroalkyl, C₅-C₂₀ heteroaryl, heteroatom-containing C₅-C₃₀ aralkyl, or heteroatom-containing C₅-C₃₀ alkaryl). Additionally, two or more of R⁵, R⁶, R⁹, and R¹⁰ can be taken together to form a cyclic group, which may be, for example, five- or six-membered rings, or two or three five- or six-membered rings, which may be either fused or linked. The cyclic groups may be aliphatic or aromatic, and may be heteroatom-containing and/or substituted.

The C₂ to C₅₀ cyclic olefins may be strained or unstrained, monocyclic or polycyclic, and may optionally include heteroatoms and or one or more functional groups. Exemplary monocyclic olefins represented by Formula (II) (e.g., olefins wherein R⁵ and R¹⁰ are not linked) may include, but are not limited to, cyclopentene, 3-methylcyclopentene, 4-methylcyclopentene, 3-t-butyldimethylsilyloxycyclopentene, 4-t-butyl-dimethylsilyloxycyclopentene, cyclohexene, 3-methylcyclohexene, 4-methyl-cyclohexene, 3-t-butyldimethylsilyloxycyclohexene, 4-t-butyldimethylsilyloxycyclohexene, cycloheptene, 3-methylcycloheptene, 4-methylcycloheptene, 5-methylcycloheptene, 3-t-butyldimethylsilyloxycycloheptene, 4-t-butyldimethylsilyloxycycloheptene, 5-t-butyldimethylsilyloxycycloheptene, cyclooctene, 3-methylcyclooctene, 4-methylcyclooctene, 5-methylcyclooctene, 3-t-butyldimethyl-silyloxycyclooctene, 4-t-butyldimethylsilyloxycyclooctene, 5-t-butyldimethylsilyloxycyclooctene, cyclononene, 3-methylcyclononene, 4-methylcyclononene, 5-methylcyclononene, 6-methylcyclo-nonene, 3 -t-butyldimethylsilyloxycyclononene, 4-t-butyldimethylsilyloxycyclononene, 5-t-butyl-dimethylsilyloxycyclononene, 6-t-butyldimethylsilyloxycyclononene, cyclodecene, 3-methylcyclo-decene, 4-methylcyclodecene, 5-methylcyclodecene, 6-methylcyclodecene, 3 -t-butyldimethylsilyloxycyclodecene, 4-t-butyldimethylsilyloxycyclononene, 5-t-butyldimethylsilyloxycyclodecene, 6-t-butyldimethylsilyloxycyclodecene, cycloundecene, 3-methylcycloundecene, 4-methylcycloundecene, 5-methylcycloundecene, 6-methylcycloundecene, 7-methylcycloundecene, 3 -t-butyldimethylsilyloxycycloundecene, 4-t-butyldimethylsilyloxycycloundecene, 5-t-butyldimethylsilyloxy-cycloundecene, 6-t-butyldimethylsilyloxycycloundecene, 7-t-butyldimethylsilyloxycycloundecene, cyclododecene, 3 -methylcyclododecene, 4-methylcyclododecene, 5-methylcyclododecene, 6-methyl-cyclododecene, 7-methylcyclododecene, 3-t-butyldimethylsilyloxycyclododecene, 4-t-butyldimethylsilyloxycyclododecene, 5-t-butyldimethylsilyloxycyclododecene, 6-t-butyldimethylsilyloxycyclododecene, and 7-t-butyldimethylsilyloxycyclododecene.

Non-limiting examples of cyclic olefins and diolefins may include cyclopropene, cyclobutene, cyclopentene, cyclohexene, cycloheptene, cyclooctene, cyclononene, cyclodecene, norbornene, 4-methylnorbornene, 7-oxanorbornene, 2-methylcyclopentene, 4-methylcyclopentene, vinylcyclohexane, 5-ethylidene-2-norbornene, vinylcyclohexene, 5-vinyl-2-norbornene, 1,3-divinylcyclopentane, 1,2-divinylcyclohexane, 1,3-divinylcyclohexane, 1,4-divinylcyclohexane, 1,5-1,5-divinylcyclooctane, 1-allyl-4-vinylcyclohexane, 1,4-diallylcyclohexane, 1-allyl-5-vinylcyclooctane, and 1,5-diallylcyclooctane.

Furthermore, examples of dienes (cyclic and acyclic) may include alpha-omega-dienes (e.g., di-vinyl monomers), butadiene, pentadiene, hexadiene, heptadiene, octadiene, nonadiene, decadiene, undecadiene, dodecadiene, tridecadiene, tetradecadiene, pentadecadiene, hexadecadiene, heptadecadiene, octadecadiene, nonadecadiene, icosadiene, heneicosadiene, docosadiene, tricosadiene, tetracosadiene, pentacosadiene, hexacosadiene, heptacosadiene, octacosadiene, nonacosadiene, triacontadiene, for example dienes include 1,6-heptadiene, 1,7-octadiene, 1,8-nonadiene, 1,9-decadiene, 1,10-undecadiene, 1,11-dodecadiene, 1,12-tridecadiene, 1,13-tetradecadiene, methyl-cyclopentadiene, cyclooctadiene, 1,5-cyclooctadiene, norbornadiene, vinylnorbornene, divinylbenzene, 7-oxanorbornadiene, 5-ethylidene-2-norbornene, 5-vinyl-2-norbornene, divinylbenzene, 1,4-hexadiene, 5-methylene-2-norbornene, 1,6-octadiene, 5-methyl-1,4-hexadiene, 3,7-dimethyl-1,6-octadiene, 1,3-cyclopentadiene, 1,4-cyclohexadiene, dicyclopentadiene, substituted derivatives thereof, and isomers thereof, or higher ring containing diolefins with or without substituents at various ring positions.

For example, the cyclic olefin monomer represented by formulae (II), (IV), and (V) can be a vinylenes, such as an olefin with a “cis-” conformation, such as an olefin with “trans-” conformation, or a mixture thereof, thus at any proportion thereof. Furthermore, the cyclic olefin monomer can be a tri-substituted vinylene. Traces of tetra-substituted vinylene may be present in the reaction mixture.

The C₂-C₅₀ acyclic olefins of Formula (I) (such as C₂-C₂₀ acyclic olefins, such as C₉-C₁₁ acyclic olefins) can be produced with a weight average molecular weight (Mw) of from about 28 g/mol to about 700 g/mol, such as from about 28 g/mol to about 420 g/mol, such as from about 28 g/mol to about 280 g/mol. The C₃-C₅₀ cyclic olefins of Formula (II) can be produced with a weight average molecular weight (Mw) of from about 40 g/mol to about 698 g/mol, such as from about 40 g/mol to about 418 g/mol, such as from about 40 g/mol to about 278 g/mol.

### Polymerizing the Olefin Products

The olefin products of Formula (I) and Formula (II) are polymerized (e.g., copolymerized) to form products. For example, olefin metathesis allows the substituents of different olefins to rearrange into new olefins, and therefore form new products. When two acyclic olefins undergo an olefin metathesis insertion reaction, two new olefins are formed; therefore the molecular weight growth is limited. However, the present disclosure provides a process such that a cyclic olefin and an acyclic olefin can be polymerized together (e.g., copolymerized), which gives one product having the sum of carbon numbers of the feed molecules. For example, ring opening metathesis of the cyclic olefins can lead to polymers (e.g., Ring Opening Metathesis Polymerization, ROMP), such that larger molecular weight molecules can also be formed. The olefins feed may include: i) strained cyclic olefins (e.g., cyclopentene, norbornene); ii) a mixture of two or more feeds, for example, a reaction between a mixture of C₅ olefins (e.g., pentenes including cyclopentene) may result in the formation of C₁₀ and C₁₅ products, thus due to the cyclopentene polymerization. Accordingly, the average molecular weight of the olefin products can be controlled by adjusting: i) the cyclic/acyclic olefin ratio; ii) the catalyst formulation; and/or iii) having secondary reactions to further break up larger molecules (e.g., ethenolysis reactions using cross metathesis with ethylene to break up larger olefin molecules), and unreactive cyclic olefins (e.g., cyclohexene) can be used to form oligomers.

For example, ethenolysis is a catalytic process, such as a cross metathesis, that can convert higher molecular weight internal alkenes to more valuable terminal alkenes (e.g., alpha-olefins) by degrading such internal olefins using ethylene as a reagent. For example, the ethenolysis can be employed to produce diolefins, e.g., α,ω-dienes, by reacting a cyclic alkene with ethylene, in presence of a transition metal catalyst suitable for cross metathesis (e.g., Rhenium(VII) oxide supported on alumina). Furthermore, the resulting terminal alkenes (e.g., alpha-olefins) can be subject to an oligomerization process in the presence of a Lewis acid catalyst, such as BF₃, thus producing polyalphaolefins (PAOs) base stocks, such as Group IV base stocks. In this regard, a recycle process involving a dehydrogenation process performed on a polymer, such as polyethylene, followed by an olefin metathesis process, can lead to fragmentation of the polymer. Also, the conversion to terminal alkenes (e.g., alpha-olefins) can yield the formation of vinyl terminated monomers, which can be used for mold-making elastomers or encapsulants/sealants for electronic components, for instance.

The present disclosure provides a method for synthesizing an olefinic polymer, such as C₅-C₂₀₀ olefins (III), such as C₅-C₁₀₀ olefins (III), using a ROMP reaction, comprising contacting an olefin monomer with a catalytically effective amount of an olefin metathesis catalyst under reaction conditions effective to allow the ROMP reaction to occur, wherein the olefin monomer contains a plurality of heteroatoms, at least two of which are directly or indirectly linked to each other. By “directly” linked is meant that the two heteroatoms are linked to each other through a direct, covalent bond. By “indirectly” linked is meant that one or more atoms are present between the heteroatoms. For example, the “indirect” linkage herein refers to the presence of a single atom (that may or may not be substituted) to which each heteroatom can be linked through a direct covalent bond. In at least one embodiment, the olefin monomer contains one double bond, and the two heteroatoms are symmetrically positioned with respect to any axis that is perpendicular to the double bond.

In at least one embodiment, a C₅-C₂₀₀ olefin, such as a C₅-C₁₀₀ olefin is represented by formula (III):

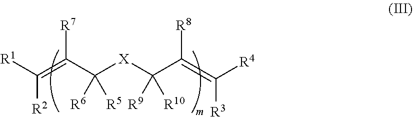

wherein:
X is a one-atom to five-atom linkage (with a “one-atom” linkage referring to a linkage that provides a single, optionally substituted atom between the two adjacent carbon atoms, and a “five-atom” linkage, similarly, referring to a linkage that provides five optionally substituted atoms between the two adjacent carbon atoms);
m is 1 to 30, such as 1 to 25, such as 1 to 20;
R¹, R², R³, and R⁴ are independently hydrogen, C₁C₄₀ hydrocarbyl (e.g., C₁-C₂₀ alkyl, C₂-C₂₀ alkenyl, C₂-C₂₀ alkynyl, C₅-C₂₀ aryl, C₆-C₂₄ alkaryl, C₆-C₂₄ aralkyl, C₁-C₂₀ alkyl, C₅-C₂₀ aryl, C₅-C₃₀ aralkyl, or C₅-C₃₀ alkaryl), C₁-C₄₀ substituted hydrocarbyl (e.g., substituted C₁-C₂₀ alkyl, C₂-C₂₀ alkenyl, C₂-C₂₀ alkynyl, C₅-C₂₀ aryl, C₆-C₂₄ alkaryl, C₆-C₂₄ aralkyl, C₁-C₂₀ alkyl, C₅-C₂₀ aryl, C₅-C₃₀ aralkyl, or C₅-C₃₀ alkaryl), a heteroatom or a heteroatom-containing group, such as each of R², R³, and R⁴ is independently selected from hydrogen, methyl, ethyl, propyl, butyl, pentyl, hexyl, heptyl, octyl, nonyl, decyl, undecyl, dodecyl, phenyl, substituted phenyl, biphenyl or an isomer thereof, which may be halogenated (such as bromopropyl, bromopropyl, bromobutyl, (bromomethyl)cyclopropyl, chloroethyl, 2,3,5,6-tetrafluorobenzyl, perfluoropropyl, perfluorobutyl, perfluoroethyl, perfluoromethyl), substituted hydrocarbyl radicals and isomers of substituted hydrocarbyl radicals such as trimethylsilylpropyl, trimethylsilylmethyl, trimethylsilylethyl, phenyl, or isomers of hydrocarbyl substituted phenyl such as methylphenyl, dimethylphenyl, trimethylphenyl, tetramethylphenyl, pentamethylphenyl, diethylphenyl, triethylphenyl, propylphenyl, dipropylphenyl, tripropylphenyl, dimethylethylphenyl, dimethylpropylphenyl, dimethylbutylphenyl, and dipropylmethylphenyle, such as R² and R³ are independently hydrogen or C₁-C₄₀ hydrocarbyl (e.g., C₁-C₂₀ alkyl, C₂-C₂₀ alkenyl, C₂-C₂₀ alkynyl, C₅-C₂₀ aryl, C₆-C₂₄ alkaryl, C₆-C₂₄ aralkyl, C₁-C₂₀ alkyl, C₅-C₂₀ aryl, C₅-C₃₀ aralkyl, or C₅-C₃₀ alkaryl), substituted hydrocarbyl (e.g., substituted C₁-C₂₀ alkyl, C₂-C₂₀ alkenyl, C₂-C₂₀ alkynyl, C₅-C₂₀ aryl, C₆-C₂₄ alkaryl, C₆-C₂₄ aralkyl, C₁-C₂₀ alkyl, C₅-C₂₀ aryl, C₅-C₃₀ aralkyl, or C₅-C₃₀ alkaryl), such as methyl, ethyl, propyl, butyl, pentyl, hexyl, heptyl, octyl, nonyl, decyl, undecyl, dodecyl, phenyl, substituted phenyl, biphenyl or an isomer thereof, such as perfluoropropyl-, perfluorobutyl-, perfluoroethyl-, or perfluoromethyl-substituted hydrocarbyl radicals and isomers of substituted hydrocarbyl radicals such as trimethylsilylpropyl, trimethylsilylmethyl, trimethylsilylethyl, or phenyl, and isomers of hydrocarbyl substituted phenyl such as methylphenyl, dimethylphenyl, trimethylphenyl, tetramethylphenyl, pentamethylphenyl, diethylphenyl, triethylphenyl, propylphenyl, dipropylphenyl, tripropylphenyl, dimethylethylphenyl, dimethylpropylphenyl, dimethylbutylphenyl, and dipropylmethylphenyl; and R¹ and R⁴ are independently selected from hydrogen, methyl, ethyl, propyl, butyl, pentyl, hexyl, heptyl, octyl, nonyl, decyl, undecyl, dodecyl, phenyl, substituted phenyl, biphenyl or an isomer thereof, which may be halogenated (such as bromopropyl, bromopropyl, bromobutyl, (bromomethyl)cyclopropyl, chloroethyl, 2,3,5,6-tetrafluorobenzyl, perfluoropropyl, perfluorobutyl, perfluoroethyl, perfluoromethyl), substituted hydrocarbyl radicals and isomers of substituted hydrocarbyl radicals such as trimethylsilylpropyl, trimethylsilylmethyl, trimethylsilylethyl, phenyl, or isomers of hydrocarbyl substituted phenyl such as methylphenyl, dimethylphenyl, trimethylphenyl, tetramethylphenyl, pentamethylphenyl, diethylphenyl, triethylphenyl, propylphenyl, dipropylphenyl, tripropylphenyl, dimethylethylphenyl, dimethylpropylphenyl, dimethylbutylphenyl, and dipropylmethylphenyl, such as R² and R³ are hydrogen and R¹ and R⁴ are independently selected from hydrogen, methyl, ethyl, propyl, butyl, pentyl, hexyl, heptyl, octyl, nonyl, decyl, undecyl, dodecyl, phenyl, substituted phenyl, biphenyl or an isomer thereof, which may be halogenated (such as bromopropyl, bromopropyl, bromobutyl, (bromomethyl)cyclopropyl, chloroethyl, 2,3,5,6-tetrafluorobenzyl, perfluoropropyl, perfluorobutyl, perfluoroethyl, or perfluoromethyl), substituted hydrocarbyl radicals and isomers of substituted hydrocarbyl radicals such as trimethylsilylpropyl, trimethylsilylmethyl, trimethylsilylethyl, phenyl, or isomers of hydrocarbyl substituted phenyl such as methylphenyl, dimethylphenyl, trimethylphenyl, tetramethylphenyl, pentamethylphenyl, diethylphenyl, triethylphenyl, propylphenyl, dipropylphenyl, tripropylphenyl, dimethylethylphenyl, dimethylpropylphenyl, dimethylbutylphenyl, and dipropylmethylphenyl;
one of Wand R⁸ is hydrogen and the other is selected from hydrogen, hydrocarbyl (e.g., C₁-C₂₀ alkyl, C₂-C₂₀ alkenyl, C₂-C₂₀ alkynyl, C₅-C₂₀ aryl, C₆-C₂₄ alkaryl, and C₆-C₂₄ aralkyl), substituted hydrocarbyl (e.g., substituted C₁-C₂₀ alkyl C₂-C₂₀ alkenyl, C₂-C₂₀ alkynyl, C₅-C₂₀ aryl, C₆-C₂₄ alkaryl , and C₆-C₂₄ aralkyl), heteroatom-containing hydrocarbyl (e.g., heteroatom-containing C₁-C₂₀ alkyl, C₂-C₂₀ alkenyl, C₂-Cz₂₀ alkynyl, C₅-C₂₀ aryl, C₆-C₂₄ alkaryl, and C₆-C₂₄ aralkyl), or substituted heteroatom-containing hydrocarbyl (e.g., substituted heteroatom-containing C₁-C₂₀ alkyl, C₂-C₂₀ alkenyl, C₂-C₂₀ alkynyl, C₅-C₂₀ aryl, C₆-C₂₄ alkaryl, and C₆-C₂₄ aralkyl); and
R⁵, R⁶, R⁹, and R¹⁰ are independently selected from hydrogen, hydrocarbyl, substituted hydrocarbyl, heteroatom-containing hydrocarbyl, and substituted heteroatom-containing hydrocarbyl, and further wherein any two of R⁵, R⁶, R⁹, and R¹⁰ may be taken together to form a cyclic structure, such that the olefin monomer is bicyclic and X can be a one-atom or two-atom linkage.

In at least one embodiment, R⁵, R⁶, R⁹, and R¹⁰ are independently selected from hydrogen, hydrocarbyl (e.g., C₁-C₂₀ alkyl, C₅-C₂₀ aryl, C₅-C₃₀ aralkyl, or C₅-C₃₀ alkaryl), substituted hydrocarbyl (e.g., substituted C₁-C₂₀ alkyl, C₅-C₂₀ aryl, C₅-C₃₀ aralkyl, or C₅-C₃₀ alkaryl), heteroatom-containing hydrocarbyl (e.g., C₁-C₂₀ heteroalkyl, C₅-C₂₀ heteroaryl, heteroatom-containing C₅-C₃₀ aralkyl, or heteroatom-containing C₅-C₃₀ alkaryl), substituted heteroatom-containing hydrocarbyl (e.g., substituted C₁-C₂₀ heteroalkyl, C₅-C₂₀ heteroaryl, heteroatom-containing C₅-C₃₀ aralkyl, or heteroatom-containing C₅-C₃₀ alkaryl). Additionally, any two or more of R⁵, R⁶, R⁹, and R¹⁰ can be taken together to form a cyclic group, which may be, for example, five- or six-membered rings, or two or three five- or six-membered rings, which may be either fused or linked. The cyclic groups may be aliphatic or aromatic, and may be heteroatom-containing and/or substituted.

One group of such cyclic olefins are those of formula (II) wherein R⁶ and R¹⁰ are hydrogen, R⁵ is -(Q)ₖ-E wherein k is zero and -E is —Y—(R¹⁶)ₙ, and R⁹ is -(Q)ₖ-E wherein k is zero and E is —Z—(R¹⁷)ⱼ, with Y and Z are independently N, O, or S, k is zero or 1, j and n are independently zero or 1, Q is a one-atom to five-atom linkage. In at least one embodiment, and when the monomer is bicyclic (e.g., when R¹⁶ and R¹⁷ are linked), then Q is a one-atom or two-atom linkage, such as a linkage that has one or two optionally substituted atoms between the two carbon atoms to which Q is bound. For example, Q can be of the formula —CR¹¹R¹²′—(Q¹)_{q}′— wherein q′ is zero or 1, Q¹ is CR¹³R¹⁴′, O, S, or NR¹⁵′, and R¹¹′, R¹²′, R¹³′, R¹⁴′, and R^{15′} are independently selected from hydrogen, hydrocarbyl (e.g., C₁-C₂₀ alkyl, C₂-C₂₀ alkenyl, C₂-C₂₀ alkynyl, C₅-C₂₀ aryl, C₆-C₂₄ alkaryl, C₆-C₂₄ aralkyl, C₁-C₂₀ alkyl, C₅-C₂₀ aryl, C₅-C₃₀ aralkyl, or C₅-C₃₀ alkaryl), substituted hydrocarbyl (e.g., substituted C₁-C₂₀ alkyl, C₂-C₂₀ alkenyl, C₂-C₂₀ alkynyl, C₅-C₂₀ aryl, C₆-C₂₄ alkaryl, C₆-C₂₄ aralkyl, C₁C₂₀ alkyl, C₅-C₂₀ aryl, C₅-C₃₀ aralkyl, or C₅-C₃₀ alkaryl), heteroatom-containing hydrocarbyl (e.g., C₁-C₂₀ heteroalkyl, C₅-C₂₀ heteroaryl, heteroatom-containing C₅-C₃₀ aralkyl, or heteroatom-containing C₅-C₃₀ alkaryl), substituted heteroatom-containing hydrocarbyl (e.g., substituted C₁-C₂₀ heteroalkyl, C₅-C₂₀ heteroaryl, heteroatom-containing C₅-C₃₀ aralkyl, or heteroatom-containing C₅-C₃₀ alkaryl); When q′ is 1, suitable examples of linkages can be wherein Q¹ is CR¹³′R¹⁴′, thus providing a substituted or unsubstituted ethylene moiety to the cyclic olefin of Formula (IV). Accordingly, when R¹¹′, R¹²′, R^{13′}, and R¹⁴′ are hydrogen, then Q is ethylene. When q′ is zero, the linkage can be substituted or unsubstituted methylene, and a suitable linkage within this group can be methylene (e.g., when R¹¹′ and R¹²′ are both hydrogen; and R¹⁶ and R¹⁷ are independently selected from hydrogen, hydrocarbyl, substituted hydrocarbyl, heteroatom-containing hydrocarbyl, substituted heteroatom-containing hydrocarbyl, and amino groups, wherein R¹⁶ and R¹⁷ may be taken together to form a cyclic group, and further wherein Y and Z are directly or indirectly linked. In at least one embodiment, the cyclic olefin monomer is represented by formula (IV):

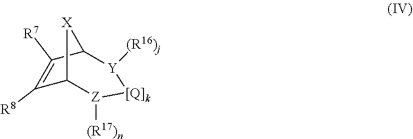

wherein:
X is a one-atom to five-atom linkage. In at least one embodiment, and when the monomer is bicyclic (e.g., when R⁵ and R¹⁰ are linked), then X is a one-atom or two-atom linkage, such as a linkage that has one or two optionally substituted atoms between the two carbon atoms to which Xis bound. For example, X can be of the formula —CR¹¹R¹²—(X¹)_{q}— wherein q is zero or 1, X¹ is CR¹³R¹⁴, O, S, or NR¹⁵, and R¹¹, R¹², R¹³, R¹⁴, and R¹⁵ are independently selected from hydrogen, hydrocarbyl (e.g., C₁-C₂₀ alkyl, C₂-C₂₀ alkenyl, C₂-C₂₀ alkynyl, C₅-C₂₀ aryl, C₆-C₂₄ alkaryl, C₆-C₂₄ aralkyl, C₁-C₂₀ alkyl, C₅-C₂₀ aryl, C₅-C₃₀ aralkyl, or C₅-C₃₀ alkaryl), substituted hydrocarbyl (e.g., substituted C₁-C₂₀ alkyl, C₂-C₂₀ alkenyl, C₂-C₂₀ alkynyl, C₅-C₂₀ aryl, C₆-C₂₄ alkaryl, C₆-C₂₄ aralkyl, C₁-C₂₀ alkyl, C₅-C₂₀ aryl, C₅-C₃₀ aralkyl, or C₅-C₃₀ alkaryl), heteroatom-containing hydrocarbyl (e.g., C₁-C₂₀ heteroalkyl, C₅-C₂₀ heteroaryl, heteroatom-containing C₅-C₃₀ aralkyl, or heteroatom-containing C₅-C₃₀ alkaryl), substituted heteroatom-containing hydrocarbyl (e.g., substituted C₁-C₂₀ heteroalkyl, C₅-C₂₀ heteroaryl, heteroatom-containing C₅-C₃₀ aralkyl, or heteroatom-containing C₅-C₃₀ alkaryl).
When q is 1, suitable examples of linkages can be wherein X¹ is CR¹³R¹⁴, thus providing a substituted or unsubstituted ethylene moiety. Accordingly, when R¹¹, R¹², R¹³, and R¹⁴ are hydrogen, then X is ethylene. When q is zero, the linkage can be substituted or unsubstituted methylene, and a suitable linkage within this group can be methylene (e.g., when R¹¹ and R¹² are both hydrogen);
one of R⁷ and R⁸ is hydrogen and the other is selected from hydrogen, hydrocarbyl (e.g., C₁-C₂₀ alkyl, C₂-C₂₀ alkenyl, C₂-C₂₀ alkynyl, C₅-C₂₀ aryl, C₆-C₂₄ alkaryl, and C₆-C₂₄ aralkyl), substituted hydrocarbyl (e.g., substituted C₁-C₂₀ alkyl, C₂-C₂₀ alkenyl, C₂-C₂₀ alkynyl, C₅-C₂₀ aryl, C₆-C₂₄ alkaryl, and C₆-C₂₄ aralkyl), heteroatom-containing hydrocarbyl (e.g., heteroatom-containing C₁-C₂₀ alkyl, C₂-C₂₀ alkenyl, C₂-C₂₀ alkynyl, C₅-C₂₀ aryl, C₆-C₂₄ alkaryl, and C₆-C₂₄ aralkyl), or substituted heteroatom-containing hydrocarbyl (e.g., substituted heteroatom-containing C₁-C₂₀ alkyl, C₂-C₂₀ alkenyl, C₂-C₂₀ alkynyl, C₅-C₂₀ aryl, C₆-C₂₄ alkaryl, and C₆-C₂₄ aralkyl); Y and Z are independently N, O, or S;
k is zero or 1;
j and n are independently zero or 1;
Q is a one-atom to five-atom linkage. In at least one embodiment, and when the monomer is bicyclic (e.g., when R¹⁶ and R¹⁷ are linked), then Q is a one-atom or two-atom linkage, such as a linkage that has one or two optionally substituted atoms between the two carbon atoms to which Q is bound. For example, Q can be of the formula —CR¹¹R¹²′—(Q¹)_{q}′— wherein q′ is zero or 1, Q¹ is CR¹³′R¹⁴′, O, S, or NR¹⁵′, and R¹¹′, R¹³′, R¹³′, R¹⁴′, and R¹⁵′ are independently selected from hydrogen, hydrocarbyl (e.g., C₁-C₂₀ alkyl, C₂-C₂₀ alkenyl, C₂-C₂₀ alkynyl, C₅-C₂₀ aryl, C₆-C₂₄ alkaryl, C₆-C₂₄ aralkyl, C₁-C₂₀ alkyl, C₅-C₂₀ aryl, C₅-C₃₀ aralkyl, or C₅-C₃₀ alkaryl), substituted hydrocarbyl (e.g., substituted C₁-C₂₀ alkyl, C₂-C₂₀ alkenyl, C₂-C₂₀ alkynyl, C₅-C₂₀ aryl, C₆-C₂₄ alkaryl, C₆-C₂₄ aralkyl, C₁-C₂₀ alkyl, C₅-C₂₀ aryl, C₅-C₃₀ aralkyl, or C₅-C₃₀ alkaryl), heteroatom-containing hydrocarbyl (e.g., C₁-C₂₀ heteroalkyl, C₅-C₂₀ heteroaryl, heteroatom-containing C₅-C₃₀ aralkyl, or heteroatom-containing C₅-C₃₀ alkaryl), substituted heteroatom-containing hydrocarbyl (e.g., substituted C₁-C₂₀ heteroalkyl, C₅-C₂₀ heteroaryl, heteroatom-containing C₅-C₃₀ aralkyl, or heteroatom-containing C₅-C₃₀ alkaryl); When q′ is 1, suitable examples of linkages can be wherein Q¹ is CR¹³′R¹⁴′, thus providing a substituted or unsubstituted ethylene moiety to the cyclic olefin of Formula (IV). Accordingly, when R¹¹′, R¹²′, R¹³′, and R¹⁴′ are hydrogen, then Q is ethylene. When q′ is zero, the linkage can be substituted or unsubstituted methylene, and a suitable linkage within this group can be methylene (e.g., when R¹¹′ and R¹²′ are both hydrogen);
R¹⁶ and R¹⁷ are independently selected from hydrogen, hydrocarbyl, substituted hydrocarbyl, heteroatom-containing hydrocarbyl, substituted heteroatom-containing hydrocarbyl, and amino groups, wherein R¹⁶ and R¹⁷ may be taken together to form a cyclic group;
when Y is O or S, then n is zero;
when Z is O or S, then j is zero;
when Y is N, then n is 1; and
when Z is N, then j is 1.

In at least one embodiment, forming the products from the cyclic olefin(s) and acyclic olefin(s), e.g., by ring-opening cross-metathesis, such as ROMP, is performed at a cyclic/acyclic olefin molar ratio of from about 1:1000 to about 1000:1, such as from about 1:700 to about 700:1, such as from about 1:500 to about 500:1, such as from about 1:250 to about 250:1, such as from about 1:100 to about 100:1, such as from about 1:50 to about 50:1, such as from about 1:10 to about 10:1; and/or using, for example, a group 6, 7, or 8 transition metal catalyst complex (e.g., Re, Ru, Os, Mo, W), and/or any suitable heterogeneous metathesis catalysts including Re, Mo, or W oxides (further details on the catalyst will be described in the next section).

Any solvent suitable for metathesis reactions may be utilized in the present disclosure. Suitable conditions for performing provided processes may use one or more solvents. For example, the ring-opening cross-metathesis may occur in one or more organic solvents. Examples of such organic solvents include, but are not limited to, hydrocarbons such as benzene, toluene, and pentane, halogenated hydrocarbons such as dichloromethane, or polar aprotic solvents, such as ethereal solvents including ether, DME, tetrahydrofuran (THF), or dioxanes, or protic solvents, such as alcohols, or mixtures thereof. In at least one embodiment, one or more solvents are deuterated. Furthermore, examples of solvents that may be used in the ring-opening cross-metathesis reaction may include organic, polar aprotic, protic, or aqueous solvents that are inert under the ring-opening cross-metathesis conditions, such as aromatic hydrocarbons, chlorinated hydrocarbons, ethers, aliphatic hydrocarbons, alcohols, water, or mixtures thereof. Suitable organic solvents for ring-opening cross-metathesis can be, but are not limited to, dichloromethane, dichloroethane, toluene, benzene, acetonitrile, p-xylene, methylene chloride, 1,2-dichloroethane, dichlorobenzene, chlorobenzene, tetrahydrofuran, diethylether, pentane, methanol, ethanol, water, or mixtures thereof, such as the solvent can be benzene, toluene, xylenes, methylene chloride, 1,2-dichloroethane, dichlorobenzene, chlorobenzene, tetrahydrofuran, diethylether, pentane, methanol, or ethanol. In at least one embodiment, the solvent is toluene or 1,2-dichloroethane. The solubility of the C₅-C₂₀₀ olefins, such as the C₅-C₁₀₀ olefins (III) formed in the ring-opening cross-metathesis reaction will depend on the choice of solvent and the molecular weight of the C₅-C₂₀₀ olefins, such as the C₅-C₁₀₀ olefins (III) obtained. Under certain circumstances, no solvent is needed. The feedstock itself can be used directly as solvent. In at least one embodiment, a single solvent is used. Alternatively, mixtures of two or more solvents are used. For example, when a solvent mixture is used, the solvent mixture can be a mixture of an ethereal solvent and a hydrocarbon. Examples of such mixtures may include, for instance, an ether/benzene mixture, or a DME/Toluene mixture. In at least one embodiment, an exemplary mixture is a DME/Toluene mixture at a DME/Toluene ratio of about 1:1. Solvent mixtures may be comprised of equal volumes of each solvent or may contain one solvent in excess of the other solvent or solvents. In at least one embodiment wherein a solvent mixture is comprised of two solvents, the solvents may be present in a ratio of about 20:1, such as about 10:1, such as about 9:1, such as about 8:1, such as about 7:1, such as about 6:1, such as about 5:1, such as about 4:1, such as about 3:1, such as about 2:1, such as about 1:1. A solvent mixture may include an ethereal solvent and a hydrocarbon, such as the solvents may be present in a ratio of about 20:1, such as about 10:1, such as about 9:1, such as about 8:1, such as about 7:1, such as about 6:1, such as about 5:1, such as about 4:1, such as about 3:1, such as about 2:1, such as about 1:1 ethereal solvent:hydrocarbon. Furthermore, a solvent mixture may include a mixture of ether and benzene in a ratio of about 5:1.

A wide range of operating temperatures (e.g., from about ambient temperature to about 400° C.) for the cross-metathesis process can be tailored, thus depending on feed and catalyst, in order to match the conditions for other reaction processes. In at least one embodiment, the ring-opening cross-metathesis process is carried out at a temperature of from about 0° C. to about 450° C., such as from about 25° C. to about 350° C., such as from about 50° C. to about 250° C., alternatively from about 0° C. to about 100° C., such as from about 25° C. to about 75° C. Furthermore, a ring-opening cross-metathesis may be carried out at reflux.

Synthesizing an olefinic polymer, such as C₅-C₂₀₀ olefins (III), such as C₅-C₁₀₀ olefins (III), using a ROMP reaction, can be performed at a pressure of from about 0.1 kPa to about 2,000 kPa, such as from about 100 kPa to about 2,000 kPa. In at least one embodiment, the ring-opening cross-metathesis process is carried out at ambient pressure. In an alternate embodiment, the ring-opening cross-metathesis process is carried out at reduced pressure. For example, the ring-opening cross-metathesis process can be performed at an absolute pressure of from about 0.1 kPa to about 5 kPa, such as from about 0.5 kPa to about 4 kPa, such as from about 1 kPa to about 3kPa. Suitable conditions may involve a reaction time of from about 5 minutes to about 120 hours, such as from about 10 minutes to about 96 hours, such as from about 20 minutes to about 48 hours, such as from about 30 minutes to about 24 hours.

Additional coupling may occur during the ring-opening cross-metathesis of C₂-C₅₀ acyclic olefins of Formula (I) and C₃-C₅₀ cyclic olefins of Formula (II) such as a homocoupling reaction between two or more C₂-C₅₀ acyclic olefins of Formula (I) or a homocoupling reaction between two or more C₃-C₅₀ cyclic olefins of Formula (II) (e.g., cyclopentene), and the coupling reaction can be controlled via the cyclic/acyclic olefin molar ratio, for example. Interestingly, the acyclic olefins can act as chain transfer agents to regulate the molecular weight of polymers produced.

### Ring-Opening Cross-Metathesis Catalysts

In at least one embodiment, the ring-opening cross-metathesis, such as ROMP, is performed using a transition metal catalyst complex, such as a group 6, 7, or 8 transition metal catalyst complex (e.g., Re, Ru, Os, Mo, W), and/or any suitable heterogeneous metathesis catalysts including Re, Mo, or W oxides, for example.

In at least one embodiment, the ring-opening cross-metathesis, such as ROMP, is performed using a soluble group 6, 7 or 8 transition metal catalyst complex. The ring-opening cross-metathesis catalyst can be used at a catalyst loading of from about 0.001 mol % to about 10 mol %, such as from about 0.001 mol % to about 5 mol %, such as from about 0.001 mol % to about 1 mol %. High molecular weight olefins (e.g., ≥300,000 g/mol) can be achieved with a molar ratio of feed to catalyst of 500:1 or greater, such as 600:1 or greater, such as 700:1 or greater.

The ROMP reaction can be carried out in an inert atmosphere by dissolving a catalytically effective amount of an olefin metathesis catalyst, such as a group 8 transition metal complex of Formula (A), in a solvent, and adding the C₃-C₅₀ cyclic olefin monomer (such as a monomer of Formula (II)), optionally dissolved in a solvent, to the catalyst solution. In at least one embodiment, the reaction is agitated (e.g., stirred). The progress of the reaction can be monitored by standard techniques, e.g., nuclear magnetic resonance spectroscopy and/or GC analysis. For example, metathesis catalysts used for purposes of the present disclosure can be any suitable metathesis catalyst, such as a Grubbs-type catalyst or Schrock-type catalyst, such as a second generation Grubbs catalyst including Ru (Scheme 1, structure A), or Schrock-type catalyst including Mo (Scheme 1, structure B), or heterogeneous catalysts including Re, Mo, or W oxides to allow for facile use of flow reactors. Examples of suitable metathesis catalysts are described in U.S. Pat. No. 6,803,429 B2; Grubbs, R., “Handbook of Metathesis”, vol. 3, 2003, Wiley-VCH, Weinheim; Schrock et al. “Preparation and Reactivity of Several Alkylidene Complexes of the Type W(CHR′)N-2,6-C₆H₃-i-Pr₂)(OR)₂ and Related Tungstacyclobutane Complexes. Controlling Metathesis Activity through the Choice of Alkoxide Ligand” J. Am. Chem. Soc., 1988, 110, pp 1423-1435; Chabanas, et al. “A Highly Active Well-Defined Rhenium Heterogenous Catalyst for Olefin Metathesis Prepared via Surface Organometallic Chemistry” J. Am. Chem. Soc., 2001, 123, pp 2062-2063, which are incorporated by reference herein.

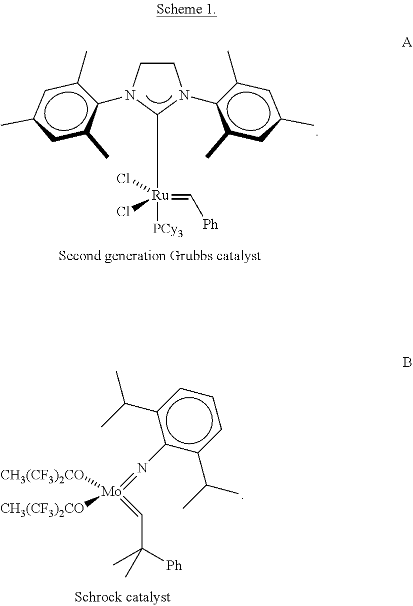

Alternatively, the ROMP reaction can be carried out in an inert atmosphere by dissolving a catalytically effective amount of an olefin metathesis catalyst, such as a group 8 transition metal complex of Formula (B)) in a solvent, and adding the C₃-C₅₀ cyclic olefin monomer (such as a monomer of Formula (II)), optionally dissolved in a solvent, to the catalyst solution. In at least one embodiment, the reaction is agitated (e.g., stirred). The progress of the reaction can be monitored by standard techniques, e.g., nuclear magnetic resonance spectroscopy and/or GC analysis.

In another embodiment, the ring-opening cross-metathesis, such as ROMP, is performed using a heterogeneous catalyst such as Re, Mo, or W oxide. Examples of suitable conditions for ring-opening cross-metathesis can be used as disclosed in Lwin, S.; Wachs, I. E. “Olefin Metathesis by Supported Metal Oxide Catalysts”, ACS Catal. (2014), 4, 2505-2520, which is incorporated herein by reference

### Ring-Opening Cross-Metathesis Products

The resulting C₅-C₂₀₀ olefins, such as the C₅-C₁₀₀ olefins of Formula (III) can be obtained having a weight average molecular weight (M_{w}) of from about 100 g/mol⁻¹ to about 2800 g/mol⁻¹, such as from about 100 g/mol⁻¹ to about 1400 g/mol⁻¹, such as from about 100 g/mol⁻¹ to about 500 g/mol⁻¹. For example, the C₅-C₂₀₀ olefins, such as the C₅-C₁₀₀ olefins of Formula (III) can be branched or linear polymers.

The C₅-C₂₀₀ olefins, such as the C₅-C₁₀₀ olefins (III) can be functionalized/substituted on one or more C═C bond(s) to form functionalized/substituted products containing a functional group (e.g., hydroxyl, mercapto, halogen (Cl, Br, or I), carboxylic, boryl, phosphoryl, sulfonato, nitrosyl), separated by a desired number of carbons. Examples of substitution/functionalization routes may include hydroisomerization, cyclization, aromatization, Diels-Alder [4+2] cycloaddition, alkylation, and/or hydrogenation to form functionalized C₅-C₂₀₀ products, such as functionalized C₅-C₁₀₀ products.

Furthermore, C₅-C₂₀₀ olefins (such as C₅-C₂₀₀ polyolefin products), such as C₅-C₁₀₀ olefins (such as C₅-C₁₀₀ polyolefin products) including substituted and/or non-substituted olefins, can be readily synthesized using C₃-C₅₀ cyclic olefins of Formula (IV). In at least one embodiment, a C₆-C₂₀₀ olefin, such as a C₆-C₁₀₀ olefin is represented by Formula (IV):

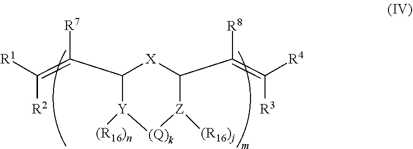

wherein:
X is a one-atom to five-atom linkage (with a “one-atom” linkage referring to a linkage that provides a single, optionally substituted atom between the two adjacent carbon atoms, and a “five-atom” linkage, similarly, referring to a linkage that provides five optionally substituted atoms between the two adjacent carbon atoms);
m is 1 to 50, such as 1 to 15, such as 1 to 5;
Y and Z are independently N, O, or S;
k is zero or 1;
j and n are independently zero or 1;
Q is a one-atom to five-atom linkage. In at least one embodiment, and when the monomer is bicyclic (e.g., when R¹⁶ and R¹⁷ are linked), then Q is a one-atom or two-atom linkage, such as a linkage that has one or two optionally substituted atoms between the two carbon atoms to which Q is bound. For example, Q can be of the formula —CR¹¹′R¹²′—(Q¹)_{q}— wherein q′ is zero or 1, Q¹ is CR¹³′R¹⁴, O, S, or NR¹⁵′, and R¹¹′, R¹²′, R¹³′, R¹⁴′, and R^{15′} are independently selected from hydrogen, hydrocarbyl (e.g., C₁-C₂₀ alkyl, C₂-C₂₀ alkenyl, C₂-C₂₀ alkynyl, C₅-C₂₀ aryl, C₆-C₂₄ alkaryl, C₆-C₂₄ aralkyl, C₁-C₂₀ alkyl, C₅-C₂₀ aryl, C₅-C₃₀ aralkyl, or C₅-C₃₀ alkaryl), substituted hydrocarbyl (e.g., substituted C₁-C₂₀ alkyl, C₂-C₂₀ alkenyl, C₂-C₂₀ alkynyl, C₅-C₂₀ aryl, C₆-C₂₄ alkaryl, C₆-C₂₄ aralkyl, C₁-C₂₀ alkyl, C₅-C₂₀ aryl, C₅-C₃₀ aralkyl, or C₅-C₃₀ alkaryl), heteroatom-containing hydrocarbyl (e.g., C₁-C₂₀ heteroalkyl, C₅-C₂₀ heteroaryl, heteroatom-containing C₅-C₃₀ aralkyl, or heteroatom-containing C₅-C₃₀ alkaryl), substituted heteroatom-containing hydrocarbyl (e.g., substituted C₁-C₂₀ heteroalkyl, C₅-C₂₀ heteroaryl, heteroatom-containing C₅-C₃₀ aralkyl, or heteroatom-containing C₅-C₃₀ alkaryl); When q′ is 1, suitable examples of linkages can be wherein Q¹ is CR¹³′R¹⁴′, thus providing a substituted or unsubstituted ethylene moiety to the cyclic olefin of Formula (IV). Accordingly, when R¹¹′, R¹²′, R^{13′}, and R¹⁴′ are hydrogen, then Q is ethylene. When q′ is zero, the linkage can be substituted or unsubstituted methylene, and a suitable linkage within this group can be methylene (e.g., when R¹¹′ and R^{12′} are both hydrogen);
R¹⁶ and R¹⁷ are independently selected from hydrogen, hydrocarbyl, substituted hydrocarbyl, heteroatom-containing hydrocarbyl, substituted heteroatom-containing hydrocarbyl, and amino groups, wherein R¹⁶ and R¹⁷ may be taken together to form a cyclic group;
when Y is O or S, then n is zero;
when Z is O or S, then j is zero;
when Y is N, then n is 1; and
when Z is N, then j is 1.

R¹, R², R³, and R⁴ are independently hydrogen, C₁-C₄₀ hydrocarbyl, C₁-C₄₀ substituted hydrocarbyl, a heteroatom or a heteroatom-containing group. In at least one embodiment, each of R¹, R², R³, and R⁴ is independently selected from hydrogen, methyl, ethyl, propyl, butyl, pentyl, hexyl, heptyl, octyl, nonyl, decyl, undecyl, dodecyl, phenyl, substituted phenyl, biphenyl or an isomer thereof, which may be halogenated (such as bromopropyl, bromopropyl, bromobutyl, (bromomethyl)cyclopropyl, chloroethyl, 2,3,5,6-tetrafluorobenzyl, perfluoropropyl, perfluorobutyl, perfluoroethyl, perfluoromethyl), substituted hydrocarbyl radicals and isomers of substituted hydrocarbyl radicals such as trimethylsilylpropyl, trimethylsilylmethyl, trimethylsilylethyl, phenyl, or isomers of hydrocarbyl substituted phenyl such as methylphenyl, dimethylphenyl, trimethylphenyl, tetramethylphenyl, pentamethylphenyl, diethylphenyl, triethylphenyl, propylphenyl, dipropylphenyl, tripropylphenyl, dimethylethylphenyl, dimethylpropylphenyl, dimethylbutylphenyl, and dipropylmethylphenyle, such as R² and R³ are independently hydrogen or C₁-C₄₀ hydrocarbyl (e.g., C₁-C₂₀ alkyl, C₂-C₂₀ alkenyl, C₂-C₂₀ alkynyl, C₅-C₂₀ aryl, C₆-C₂₄ alkaryl, C₆-C₂₄ aralkyl, C₁-C₂₀ alkyl, C₅-C₂₀ aryl, C₅-C₃₀ aralkyl, or C₅-C₃₀ alkaryl), substituted hydrocarbyl (e.g., substituted C₁-C₂₀ alkyl, C₂-C₂₀ alkenyl, C₂-C₂₀ alkynyl, C₅-C₂₀ aryl, C₆-C₂₄ alkaryl, C₆-C₂₄ aralkyl, C₁-C₂₀ alkyl, C₅-C₂₀ aryl, C₅-C₃₀ aralkyl, or C₅-C₃₀ alkaryl), such as methyl, ethyl, propyl, butyl, pentyl, hexyl, heptyl, octyl, nonyl, decyl, undecyl, dodecyl, phenyl, substituted phenyl, biphenyl or an isomer thereof, such as perfluoropropyl-, perfluorobutyl-, perfluoroethyl-, or perfluoromethyl-substituted hydrocarbyl radicals and isomers of substituted hydrocarbyl radicals such as trimethylsilylpropyl, trimethylsilylmethyl, trimethylsilylethyl, or phenyl, and isomers of hydrocarbyl substituted phenyl such as methylphenyl, dimethylphenyl, trimethylphenyl, tetramethylphenyl, pentamethylphenyl, diethylphenyl, triethylphenyl, propylphenyl, dipropylphenyl, tripropylphenyl, dimethylethylphenyl, dimethylpropylphenyl, dimethylbutylphenyl, and dipropylmethylphenyl; and R¹ and R⁴ are independently selected from hydrogen, methyl, ethyl, propyl, butyl, pentyl, hexyl, heptyl, octyl, nonyl, decyl, undecyl, dodecyl, phenyl, substituted phenyl, biphenyl or an isomer thereof, which may be halogenated (such as bromopropyl, bromopropyl, bromobutyl, (bromomethyl)cyclopropyl, chloroethyl, 2,3,5,6-tetrafluorobenzyl, perfluoropropyl, perfluorobutyl, perfluoroethyl, perfluoromethyl), substituted hydrocarbyl radicals and isomers of substituted hydrocarbyl radicals such as trimethylsilylpropyl, trimethylsilylmethyl, trimethylsilylethyl, phenyl, or isomers of hydrocarbyl substituted phenyl such as methylphenyl, dimethylphenyl, trimethylphenyl, tetramethylphenyl, pentamethylphenyl, diethylphenyl, triethylphenyl, propylphenyl, dipropylphenyl, tripropylphenyl, dimethylethylphenyl, dimethylpropylphenyl, dimethylbutylphenyl, and dipropylmethylphenyl.

In at least one embodiment, R² and R³ are hydrogen and R¹ and R⁴ are independently selected from hydrogen, methyl, ethyl, propyl, butyl, pentyl, hexyl, heptyl, octyl, nonyl, decyl, undecyl, dodecyl, phenyl, substituted phenyl, biphenyl or an isomer thereof, which may be halogenated (such as bromopropyl, bromopropyl, bromobutyl, (bromomethyl)cyclopropyl, chloroethyl, 2,3,5,6-tetrafluorobenzyl, perfluoropropyl, perfluorobutyl, perfluoroethyl, or perfluoromethyl), substituted hydrocarbyl radicals and isomers of substituted hydrocarbyl radicals such as trimethylsilylpropyl, trimethylsilylmethyl, trimethylsilylethyl, phenyl, or isomers of hydrocarbyl substituted phenyl such as methylphenyl, dimethylphenyl, trimethylphenyl, tetramethylphenyl, pentamethylphenyl, diethylphenyl, triethylphenyl, propylphenyl, dipropylphenyl, tripropylphenyl, dimethylethylphenyl, dimethylpropylphenyl, dimethylbutylphenyl, and to dipropylmethylphenyl.

One of R⁷ and R⁸ is hydrogen and the other is selected from hydrogen, hydrocarbyl (e.g., C₁-C₂₀ alkyl, C₂-C₂₀ alkenyl, C₂-C₂₀ alkynyl, C₅-C₂₀ aryl, C₆-C₂₄ alkaryl, and C₆-C₂₄ aralkyl), substituted hydrocarbyl (e.g., substituted C₁-C₂₀ alkyl, C₂-C₂₀ alkenyl, C₂-C₂₀ alkynyl, C₅-C₂₀ aryl, C₆-C₂₄ alkaryl, and C₆-C₂₄ aralkyl), heteroatom-containing hydrocarbyl (e.g., heteroatom-containing C₁-C₂₀ alkyl, C₂-C₂₀ alkenyl, C₂-C₂₀ alkynyl, C₅-C₂₀ aryl, C₆-C₂₄ alkaryl, and C₆-C₂₄ aralkyl), or substituted heteroatom-containing hydrocarbyl (e.g., substituted heteroatom-containing C₁-C₂₀ alkyl, C₂-C₂₀ alkenyl, C₂-C₂₀ alkynyl, C₅-C₂₀ aryl, C₆-C₂₄ alkaryl, and C₆-C₂₄ aralkyl).

R⁵, R⁶, R⁹, and R¹⁰ are independently selected from hydrogen, hydrocarbyl, substituted hydrocarbyl, heteroatom-containing hydrocarbyl, and substituted heteroatom-containing hydrocarbyl, and -(D)_{w}-U, and further wherein any two of R⁵, R⁶, R⁹, and R¹⁰ may be taken together to form a cyclic structure, such that the olefin monomer is bicyclic and X can be a one-atom or two-atom linkage. In at least one embodiment, R⁵, R⁶, R⁹, and R¹⁰ are independently selected from hydrogen, hydrocarbyl (e.g., C₁-C₂₀ alkyl, C₅-C₂₀ aryl, C₅-C₃₀ aralkyl, or C₅-C₃₀ alkaryl), substituted hydrocarbyl (e.g., substituted C₁-C₂₀ alkyl, C₅-C₂₀ aryl, C₅-C₃₀ aralkyl, or C₅-C₃₀ alkaryl), heteroatom-containing hydrocarbyl (e.g., C₁-C₂₀ heteroalkyl, C₅-C₂₀ heteroaryl, heteroatom-containing C₅-C₃₀ aralkyl, or heteroatom-containing C₅-C₃₀ alkaryl), substituted heteroatom-containing hydrocarbyl (e.g., substituted C₁-C₂₀ heteroalkyl, C₅-C₂₀ heteroaryl, heteroatom-containing C₅-C₃₀ aralkyl, or heteroatom-containing C₅-C₃₀ alkaryl). Additionally, any two or more of R⁵, R⁶, R⁹, and R¹⁰ can be taken together to form a cyclic group, which may be, for example, five- or six-membered rings, or two or three five- or six-membered rings, which may be either fused or linked. The cyclic groups may be aliphatic or aromatic, and may be heteroatom-containing and/or substituted.

When R⁶ and R¹⁰ are hydrogen, the C₆-C₁₀₀ olefin is represented by Formula (VII):

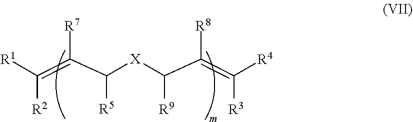

wherein:
X is a one-atom to five-atom linkage (with a “one-atom” linkage referring to a linkage that provides a single, optionally substituted atom between the two adjacent carbon atoms, and a “five-atom” linkage, similarly, referring to a linkage that provides five optionally substituted atoms between the two adjacent carbon atoms);
m is 1 to 50, such as 1 to 15, such as 1 to 5;
R¹, R², R³, and R⁴ are independently hydrogen, C₁-C₄₀ hydrocarbyl, C₁-C₄₀ substituted hydrocarbyl, a heteroatom or a heteroatom-containing group, such as each of R¹, R², R³, and R⁴ is independently selected from hydrogen, methyl, ethyl, propyl, butyl, pentyl, hexyl, heptyl, octyl, nonyl, decyl, undecyl, dodecyl, phenyl, substituted phenyl, biphenyl or an isomer thereof, which may be halogenated (such as bromopropyl, bromopropyl, bromobutyl, (bromomethyl)cyclopropyl, chloroethyl, 2,3,5,6-tetrafluorobenzyl, perfluoropropyl, perfluorobutyl, perfluoroethyl, perfluoromethyl), substituted hydrocarbyl radicals and isomers of substituted hydrocarbyl radicals such as trimethylsilylpropyl, trimethylsilylmethyl, trimethylsilylethyl, phenyl, or isomers of hydrocarbyl substituted phenyl such as methylphenyl, dimethylphenyl, trimethylphenyl, tetramethylphenyl, pentamethylphenyl, diethylphenyl, triethylphenyl, propylphenyl, dipropylphenyl, tripropylphenyl, dimethylethylphenyl, dimethylpropylphenyl, dimethylbutylphenyl, and dipropylmethylphenyle, such as R² and R³ are independently hydrogen or C₁-C₄₀ hydrocarbyl (e.g., C₁-C₂₀ alkyl, C₂-C₂₀ alkenyl, C₂-C₂₀ alkynyl, C₅-C₂₀ aryl, C₆-C₂₄ alkaryl, C₆-C₂₄ aralkyl, C₁-C₂₀ alkyl, C₅-C₂₀ aryl, C₅-C₃₀ aralkyl, or C₅-C₃₀ alkaryl), substituted hydrocarbyl (e.g., substituted C₁-C₂₀ alkyl, C₂-C₂₀ alkenyl, C₂-C₂₀ alkynyl, C₅-C₂₀ aryl, C₆-C₂₄ alkaryl, C₆-C₂₄ aralkyl, C₁-C₂₀ alkyl, C₅-C₂₀ aryl, C₅-C₃₀ aralkyl, or C₅-C₃₀ alkaryl), such as methyl, ethyl, propyl, butyl, pentyl, hexyl, heptyl, octyl, nonyl, decyl, undecyl, dodecyl, phenyl, substituted phenyl, biphenyl or an isomer thereof, such as perfluoropropyl-, perfluorobutyl-, perfluoroethyl-, or perfluoromethyl-substituted hydrocarbyl radicals and isomers of substituted hydrocarbyl radicals such as trimethylsilylpropyl, trimethylsilylmethyl, trimethylsilylethyl, or phenyl, and isomers of hydrocarbyl substituted phenyl such as methylphenyl, dimethylphenyl, trimethylphenyl, tetramethylphenyl, pentamethylphenyl, diethylphenyl, triethylphenyl, propylphenyl, dipropylphenyl, tripropylphenyl, dimethylethylphenyl, dimethylpropylphenyl, dimethylbutylphenyl, and dipropylmethylphenyl; and R¹ and R⁴ are independently selected from hydrogen, methyl, ethyl, propyl, butyl, pentyl, hexyl, heptyl, octyl, nonyl, decyl, undecyl, dodecyl, phenyl, substituted phenyl, biphenyl or an isomer thereof, which may be halogenated (such as bromopropyl, bromopropyl, bromobutyl, (bromomethyl)cyclopropyl, chloroethyl, 2,3,5,6-tetrafluorobenzyl, perfluoropropyl, perfluorobutyl, perfluoroethyl, perfluoromethyl), substituted hydrocarbyl radicals and isomers of substituted hydrocarbyl radicals such as trimethylsilylpropyl, trimethylsilylmethyl, trimethylsilylethyl, phenyl, or isomers of hydrocarbyl substituted phenyl such as methylphenyl, dimethylphenyl, trimethylphenyl, tetramethylphenyl, pentamethylphenyl, diethylphenyl, triethylphenyl, propylphenyl, dipropylphenyl, tripropylphenyl, dimethylethylphenyl, dimethylpropylphenyl, dimethylbutylphenyl, and dipropylmethylphenyl, such as R² and R³ are hydrogen and R¹ and R⁴ are independently selected from hydrogen, methyl, ethyl, propyl, butyl, pentyl, hexyl, heptyl, octyl, nonyl, decyl, undecyl, dodecyl, phenyl, substituted phenyl, biphenyl or an isomer thereof, which may be halogenated (such as bromopropyl, bromopropyl, bromobutyl, (bromomethyl)cyclopropyl, chloroethyl, 2,3,5,6-tetrafluorobenzyl, perfluoropropyl, perfluorobutyl, perfluoroethyl, or perfluoromethyl), substituted hydrocarbyl radicals and isomers of substituted hydrocarbyl radicals such as trimethylsilylpropyl, trimethylsilylmethyl, trimethylsilylethyl, phenyl, or isomers of hydrocarbyl substituted phenyl such as methylphenyl, dimethylphenyl, trimethylphenyl, tetramethylphenyl, pentamethylphenyl, diethylphenyl, triethylphenyl, propylphenyl, dipropylphenyl, tripropylphenyl, dimethylethylphenyl, dimethylpropylphenyl, dimethylbutylphenyl, and dipropylmethylphenyl;
One of R⁷ and R⁸ is hydrogen and the other is selected from hydrogen, hydrocarbyl (e.g., C₁-C₂₀ alkyl, C₂-C₂₀ alkenyl, C₂-C₂₀ alkynyl, C₅-C₂₀ aryl, C₆-C₂₄ alkaryl, and C₆-C₂₄ aralkyl), substituted hydrocarbyl (e.g., substituted C₁-C₂₀ alkyl, C₂-C₂₀ alkenyl, C₂-C₂₀ alkynyl, C₅-C₂₀ aryl, C₆-C₂₄ alkaryl, and C₆-C₂₄ aralkyl), heteroatom-containing hydrocarbyl (e.g., heteroatom-containing C₁-C₂₀ alkyl, C₂-C₂₀ alkenyl, C₂-C₂₀ alkynyl, C₅-C₂₀ aryl, C₆-C₂₄ alkaryl, and C₆-C₂₄ aralkyl), or substituted heteroatom-containing hydrocarbyl (e.g., substituted heteroatom-containing C₁-C₂₀ alkyl, C₂-C₂₀ alkenyl, C₂-C₂₀ alkynyl, C₅-C₂₀ aryl, C₆-C₂₄ alkaryl, and C₆-C₂₄ aralkyl); and
R⁵, R⁶, R⁹, and R¹⁰ may be independently selected from hydrogen, hydrocarbyl, substituted hydrocarbyl, heteroatom-containing hydrocarbyl, and substituted heteroatom-containing hydrocarbyl, and further wherein any two of R⁵, R⁶, R⁹, and R¹⁰ may be taken together to form a cyclic structure, such that the olefin monomer is bicyclic and X can be a one-atom or two-atom linkage.

In at least one embodiment, R⁵, R⁶, R⁹, and R¹⁰ are independently selected from hydrogen, hydrocarbyl (e.g., C₁-C₂₀ alkyl, C₅-C₂₀ aryl, C₅-C₃₀ aralkyl, or C₅-C₃₀ alkaryl), substituted hydrocarbyl (e.g., substituted C₁-C₂₀ alkyl, C₅-C₂₀ aryl, C₅-C₃₀ aralkyl, or C₅-C₃₀ alkaryl), heteroatom-containing hydrocarbyl (e.g., C₁-C₂₀ heteroalkyl, C₅-C₂₀ heteroaryl, heteroatom-containing C₅-C₃₀ aralkyl, or heteroatom-containing C₅-C₃₀ alkaryl), substituted heteroatom-containing hydrocarbyl (e.g., substituted C₁-C₂₀ heteroalkyl, C₅-C₂₀ heteroaryl, heteroatom-containing C₅-C₃₀ aralkyl, or heteroatom-containing C₅-C₃₀ alkaryl). Additionally, any two or more of R⁵, R⁶, R⁹, and R¹⁰ can be taken together to form a cyclic group, which may be, for example, five- or six-membered rings, or two or three five- or six-membered rings, which may be either fused or linked. The cyclic groups may be aliphatic or aromatic, and may be heteroatom-containing and/or substituted.

### Hydrogenation, Hydroisomerization and Production of Lubricants and Base Stocks

### Base Stocks

In at least one embodiment, a base stock is a C₅-C₂₀₀ polyolefin product, such as a C₅-C₁₀₀ polyolefin product, such as a C₂₅-C₅₀ polyolefin product. A hydrocarbon product of the present disclosure, when added to a lubricating oil (as a viscosity modifier) or used as a lubricating oil, can reduce the tendency of the oil to change its viscosity with temperature in order to improve its viscosity index “VI”, and flow characteristics. Improving VI helps in maintaining constant the flow properties of the protective oil film. This means a high enough viscosity to avoid damage on engine parts when the temperature rises because of the engine heat and a low enough viscosity against the cold start and pumping. Hydrocarbon products of the present disclosure, such as the C₆-C₂₀₀ olefins, such as the C₆-C₁₀₀ olefins (III), (VI), and (VII), can have a VI of about 120 or greater, such as about 140 or greater, such as about 150 or greater, such as about 170 or greater, such as about 180 or greater, as determined according to ASTM D2270.

In addition, base stocks are affected by many properties including kinematic viscosity (KV), where an inverse relationship exists between KV and low-temperature fluidity, and VI, where a direct relationship exists between VI and low-temperature fluidity. Increasing the VI of a base stock by adding a polymer product of the present disclosure can provide improved viscometrics under both low-temperature and high-temperature regimes. VI itself represents the change in viscosity over a temperature range from 40° C. to 100° C. The higher the VI, the lower the oil's viscometric properties will change, and the flatter its profile will be over the temperature range. This can be extended to higher and lower temperatures. In at least one embodiment, a polyolefin product of the present disclosure, such as the C₅-C₁₀₀ olefins (III), (VI), and (VII), can have a kinematic viscosity at 100° C. (KV100), as determined by ASTM D445, of about 20 cSt to about 200 cSt, such as from about 40 cSt to about 120 cSt, such as from about 50 cSt to about 100 cSt. Additionally or alternatively, a polyolefin product of the present disclosure such as the C₆-C₁₀₀ olefins (III), (VI), and (VII), can have a kinematic viscosity at 40° C. (KV40), as determined by ASTM D445, of about 150 cSt to about 2500 cSt, such as from about 150 cSt to about 1100 cSt.

In addition, glass transition temperature (Tg) is indicative of the fluidity of a material at low temperature operations. Tg can be measured using Differential Scanning calorimetry (DSC) on a commercially available instrument (e.g., TA Instruments 2920 DSC). Tg is measured by equilibrating the sample at 100° C., isothermal for 5 minutes, ramping the temperature at 10° C./min to −100° C., isothermal for 5 minutes, ramping the temperature at 10° C./min to 100° C., and isothermal for 2 minutes. A polyolefin product of the present disclosure, such as the C₆-C₂₀₀ olefins, such as the C₆-C₁₀₀ olefins (III), (VI), and (VII), can have a glass transition temperature (Tg) of from about −110° C. to about −50° C., such as from about −95° C. to about −75° C., such as from about -95° C. to about -85° C.

### Hydrogenation of Hydrocarbon Products

A polyolefin product, such as a C₅-C₁₀₀ olefin represented by (III), (VI), and (VII), can be catalytically hydrogenated to form a hydrogenated polyolefin product, such as a substituted or unsubstituted polymer product. A hydrogenated polyolefin product can be used as a lubricating oil base stock. The hydrogenation may be carried out in solution. The catalyst may be any suitable hydrogenation catalyst, such as a palladium catalyst supported on activated carbon or a Raney nickel catalyst. The hydrogenation can be carried out at elevated pressure, e.g., from 2,000 KPa to 10,000 KPa, such as from 4,500 KPa to 8,000 KPa. The hydrogenation reaction can be carried out at a temperature of from about 15° C. to about 400° C., such as from 50° C. to about 250° C., alternately from 30° C. to about 70° C. The duration of the hydrogenation reaction may be from a few minutes to several days. After the hydrogenation reaction is complete, the reaction mixture can be cooled, depressurized and the solvent removed by vacuum distillation. The purity of the hydrogenated product can be determined by gas chromatography, and the viscosity of the resulting lubricant can be measured by rotary viscosimetry.

The hydrogenation process can be performed with any suitable late transition metals (group 6 to 12), alloys, carbides, or nitrides that can readily hydrogenate an olefin to a corresponding saturated product;

In at least one embodiment, a hydrogenation is performed using Ni/Kiselguhr as the catalyst, at a catalyst loading of from about 1 mol % to about 5 mol %; under a pressure of from about 200 psi (1,378.95 kPa) to about 400 psi (2757.9 kPa) of hydrogen; at a temperature of from about 15° C. to about 300° C. (e.g., 250° C.); and/or a reaction time of from about 30 minutes to 4 hours.

In at least one embodiment, a hydrogenation is performed at an Hz/olefin molar ratio of from 1000:1 to 100:1; in the presence of from about 1000 mol % to about 1 mol % of Hz; and/or a gas hourly velocity (GHSV) of from about 0.01 h⁻¹ to about 1000 h⁻¹.

### Hydrogenated Polyolefin Products

Hydrogenated polyolefin products, as base stocks, produced in accordance with processes of the present disclosure can possess high linearity which can provide improved flow, low temperature properties, and thickening efficiency. Alternatively, some hydrogenated polyolefin products can be used as diesel fuels having a high cetane number.

### Hydrogenated Base Stocks

In at least one embodiment, a hydrogenated base stock is a C₅-C₂₀₀ hydrogenated polyolefin product, C₅-C₁₀₀ hydrogenated polyolefin product, such as a C₅-C₅₀ hydrogenated polyolefin product, such as a C₂₅-C₅₀ hydrogenated polyolefin product.

The high linearity of hydrogenated polyolefin products of the present disclosure can provide improved flow properties, as compared to highly branched polyolefin products. In addition, low amounts of methyl (—CH₃) moieties are also indicative of high linearity of a hydrogenated polyolefin product.

However, branched polyolefin products can be used as lubricant range products, which are of high commercial interest. Branching of said polyolefin products can be controlled using processes of the present disclosure, for example by controlling the cyclic/acyclic olefin monomer ratio and/or by using an alkyl-substituted olefin monomer suitable for the formation of a branched polyolefin product, such as an alkyl-substituted cyclic olefin and/or an alkyl-substituted acyclic olefin, such as a mono- and/or poly-alkyl substituted olefin, with at least one or more heteroatom, or without any heteroatom.

### Hydroisomerization

Hydroisomerization of the C₅-C₂₀₀ polymer products, such as the C₅-C₁₀₀ polymer products represented by Formula (VIII) or Formula (IX), such as long-chain paraffins, can be performed using one or more suitable hydroisomerization catalyst(s) (e.g., MSDW-3™ catalyst from ExxonMobil Chemical Company). Hydroisomerization of the C₅-C₁₀₀ polymers products of Formula (VIII) or Formula (IX) can reduce the content of linear paraffins in hydrocarbon mixtures by producing branched compounds, and can enable the production of high-octane gasoline and low-pour-point diesel. The hydroisomerization of the C₅-C₁₀₀ polymers products represented by formula (VIII) or Formula (IX) can improve the viscosity properties of waxy feedstocks, for example.

In at least one embodiment, the C₆-C₂₀₀ olefins, such as the C₅-C₁₀₀ olefins (III), (VI), and/or (VII), are hydrogenated to saturated products. Furthermore, the resulting saturated products formed after hydrogenation of the C₅-C₁₀₀ olefins (III), (VI), and/or (VII) can be selectively isomerized via a hydroisomerization process of such long-chain paraffins using one or more suitable hydroisomerization catalyst(s) (e.g., MSDW-3™ catalyst) to form isomerized products. Isomerized products can provide improved low temperature properties as compared to the products before isomerization.

In at least one embodiment, the C₅-C₂₀₀ olefins, such as the C₅-C₁₀₀ olefins (III), (VI), and (VII) are hydrogenated to hydrogenated polyolefin products, such as saturated products, such as C₆-C₁₀₀ polymer products represented by Formula (VIII) or Formula (IX), such as C₂₅-C₅₀ polyolefin products, which optionally may conveniently be blended with one or more other components (e.g., additives) to produce, for example, a fuel composition (e.g., higher value diesel (cetane)), waxes, lubricant range products, and base stocks.

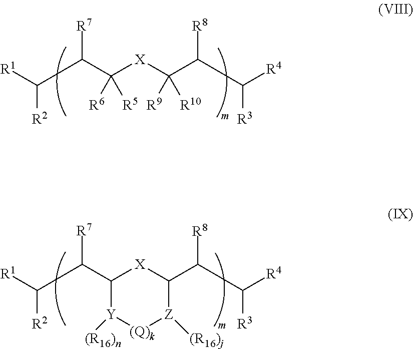

wherein:
X is a one-atom to five-atom linkage (with a “one-atom” linkage referring to a linkage that provides a single, optionally substituted atom between the two adjacent carbon atoms, and a “five-atom” linkage, similarly, referring to a linkage that provides five optionally substituted atoms between the two adjacent carbon atoms);
Q is a one-atom to five-atom linkage. In at least one embodiment, and when the monomer is bicyclic (e.g., when R¹⁶ and R¹⁷ are linked), then Q is a one-atom or two-atom linkage, such as a linkage that has one or two optionally substituted atoms between the two carbon atoms to which Q is bound. For example, Q can be of the formula —CR¹¹′R¹²′—(Q¹)_{q}′— wherein q′ is zero or 1, Q¹ is CR¹³′R14′, O, S, or NR¹⁵′, and R¹¹′, R¹²′, R¹³′, R¹⁴′, and R¹⁵′ are independently selected from hydrogen, hydrocarbyl (e.g., C₁-C₂₀ alkyl, C₂-C₂₀ alkenyl, C₂-C₂₀ alkynyl, C₅-C₂₀ aryl, C₆-C₂₄ alkaryl, C₆-C₂₄ aralkyl, C₁-C₂₀ alkyl, C₅-C₂₀ aryl, C₅-C₃₀ aralkyl, or C₅-C₃₀ alkaryl), substituted hydrocarbyl (e.g., substituted C₁-C₂₀ alkyl, C₂-C₂₀ alkenyl, C₂-C₂₀ alkynyl, C₅-C₂₀ aryl, C₆-C₂₄ alkaryl, C₆-C₂₄ aralkyl, C₁-C₂₀ alkyl, C₅-C₂₀ aryl, C₅-C₃₀ aralkyl, or C₅-C₃₀ alkaryl), heteroatom-containing hydrocarbyl (e.g., C₁-C₂₀ heteroalkyl, C₅-C₂₀ heteroaryl, heteroatom-containing C₅-C₃₀ aralkyl, or heteroatom-containing C₅-C₃₀ alkaryl), substituted heteroatom-containing hydrocarbyl (e.g., substituted C₁-C₂₀ heteroalkyl, C₅-C₂₀ heteroaryl, heteroatom-containing C₅-C₃₀ aralkyl, or heteroatom-containing C₅-C₃₀ alkaryl); When q′ is 1, suitable examples of linkages can be wherein Q¹ is CR¹³′R¹⁴′, thus providing a substituted or unsubstituted ethylene moiety to the cyclic olefin of Formula (IV). Accordingly, when R¹¹′, R¹²′, R¹³′, and R¹⁴′ are hydrogen, then Q is ethylene. When q′ is zero, the linkage can be substituted or unsubstituted methylene, and a suitable linkage within this group can be methylene (e.g., when R¹¹′ and R¹²′ are both hydrogen);
k is zero or 1;
m is 1 to 50, such as 1 to 15, such as 1 to 5;
R¹, R², R³, and R⁴ are independently hydrogen, C₁-C₄₀ hydrocarbyl, C₁-C₄₀ substituted hydrocarbyl, a heteroatom or a heteroatom-containing group, such as each of R¹, R², R³, and R⁴ is independently selected from hydrogen, methyl, ethyl, propyl, butyl, pentyl, hexyl, heptyl, octyl, nonyl, decyl, undecyl, dodecyl, phenyl, substituted phenyl, biphenyl or an isomer thereof, which may be halogenated (such as bromopropyl, bromopropyl, bromobutyl, (bromomethyl)cyclopropyl, chloroethyl, 2,3,5,6-tetrafluorobenzyl, perfluoropropyl, perfluorobutyl, perfluoroethyl, perfluoromethyl), substituted hydrocarbyl radicals and isomers of substituted hydrocarbyl radicals such as trimethylsilylpropyl, trimethylsilylmethyl, trimethylsilylethyl, phenyl, or isomers of hydrocarbyl substituted phenyl such as methylphenyl, dimethylphenyl, trimethylphenyl, tetramethylphenyl, pentamethylphenyl, diethylphenyl, triethylphenyl, propylphenyl, dipropylphenyl, tripropylphenyl, dimethylethylphenyl, dimethylpropylphenyl, dimethylbutylphenyl, and dipropylmethylphenyle, such as R² and R³ are independently hydrogen or C₁-C₄₀ hydrocarbyl (e.g., C₁-C₂₀ alkyl, C₂-C₂₀ alkenyl, C₂-C₂₀ alkynyl, C₅-C₂₀ aryl, C₆-C₂₄ alkaryl, C₆-C₂₄ aralkyl, C₁-C₂₀ alkyl, C₅-C₂₀ aryl, C₅-C₃₀ aralkyl, or C₅-C₃₀ alkaryl), substituted hydrocarbyl (e.g., substituted C₁-C₂₀ alkyl, C₂-C₂₀ alkenyl, C₂-C₂₀ alkynyl, C₅-C₂₀ aryl, C₆-C₂₄ alkaryl, C₆-C₂₄ aralkyl, C₁-C₂₀ alkyl, C₅-C₂₀ aryl, C₅-C₃₀ aralkyl, or C₅-C₃₀ alkaryl), such as methyl, ethyl, propyl, butyl, pentyl, hexyl, heptyl, octyl, nonyl, decyl, undecyl, dodecyl, phenyl, substituted phenyl, biphenyl or an isomer thereof, such as perfluoropropyl-, perfluorobutyl-, perfluoroethyl-, or perfluoromethyl-substituted hydrocarbyl radicals and isomers of substituted hydrocarbyl radicals such as trimethylsilylpropyl, trimethylsilylmethyl, trimethylsilylethyl, or phenyl, and isomers of hydrocarbyl substituted phenyl such as methylphenyl, dimethylphenyl, trimethylphenyl, tetramethylphenyl, pentamethylphenyl, diethylphenyl, triethylphenyl, propylphenyl, dipropylphenyl, tripropylphenyl, dimethylethylphenyl, dimethylpropylphenyl, dimethylbutylphenyl, and dipropylmethylphenyl; and R¹ and R⁴ are independently selected from hydrogen, methyl, ethyl, propyl, butyl, pentyl, hexyl, heptyl, octyl, nonyl, decyl, undecyl, dodecyl, phenyl, substituted phenyl, biphenyl or an isomer thereof, which may be halogenated (such as bromopropyl, bromopropyl, bromobutyl, (bromomethyl)cyclopropyl, chloroethyl, 2,3,5,6-tetrafluorobenzyl, perfluoropropyl, perfluorobutyl, perfluoroethyl, perfluoromethyl), substituted hydrocarbyl radicals and isomers of substituted hydrocarbyl radicals such as trimethylsilylpropyl, trimethylsilylmethyl, trimethylsilylethyl, phenyl, or isomers of hydrocarbyl substituted phenyl such as methylphenyl, dimethylphenyl, trimethylphenyl, tetramethylphenyl, pentamethylphenyl, diethylphenyl, triethylphenyl, propylphenyl, dipropylphenyl, tripropylphenyl, dimethylethylphenyl, dimethylpropylphenyl, dimethylbutylphenyl, and dipropylmethylphenyl, such as R² and R³ are hydrogen and R¹ and R⁴ are independently selected from hydrogen, methyl, ethyl, propyl, butyl, pentyl, hexyl, heptyl, octyl, nonyl, decyl, undecyl, dodecyl, phenyl, substituted phenyl, biphenyl or an isomer thereof, which may be halogenated (such as bromopropyl, bromopropyl, bromobutyl, (bromomethyl)cyclopropyl, chloroethyl, 2,3,5,6-tetrafluorobenzyl, perfluoropropyl, perfluorobutyl, perfluoroethyl, or perfluoromethyl), substituted hydrocarbyl radicals and isomers of substituted hydrocarbyl radicals such as trimethylsilylpropyl, trimethylsilylmethyl, trimethylsilylethyl, phenyl, or isomers of hydrocarbyl substituted phenyl such as methylphenyl, dimethylphenyl, trimethylphenyl, tetramethylphenyl, pentamethylphenyl, diethylphenyl, triethylphenyl, propylphenyl, dipropylphenyl, tripropylphenyl, dimethylethylphenyl, dimethylpropylphenyl, dimethylbutylphenyl, and dipropylmethylphenyl;
one of R⁷ and R⁸ is hydrogen and the other is selected from hydrogen, hydrocarbyl (e.g., C₁-C₂₀ alkyl, C₂-C₂₀ alkenyl, C₂-C₂₀ alkynyl, C₅-C₂₀ aryl, C₆-C₂₄ alkaryl, and C₆-C₂₄ aralkyl), substituted hydrocarbyl (e.g., substituted C₁-C₂₀ alkyl, C₂-C₂₀ alkenyl, C₂-C₂₀ alkynyl, C₅-C₂₀ aryl, C₆-C₂₄ alkaryl, and C₆-C₂₄ aralkyl), heteroatom-containing hydrocarbyl (e.g., heteroatom-containing C₁-C₂₀ alkyl, C₂-C₂₀ alkenyl, C₂-C₂₀ alkynyl, C₅-C₂₀ aryl, C₆-C₂₄ alkaryl, and C₆-C₂₄ aralkyl), or substituted heteroatom-containing hydrocarbyl (e.g., substituted heteroatom-containing C₁-C₂₀ alkyl, C₂-C₂₀ alkenyl, C₂-C₂₀ alkynyl, C₅-C₂₀ aryl, C₆-C₂₄ alkaryl, and C₆-C₂₄ aralkyl); and
R⁵, R⁶, R⁹, andR¹⁰ are independently selected from hydrogen, hydrocarbyl, substituted hydrocarbyl, heteroatom-containing hydrocarbyl, and substituted heteroatom-containing hydrocarbyl, and further wherein any two of R⁵, R⁶, R⁹, and R¹⁰ may be taken together to form a cyclic structure, such that the olefin monomer is bicyclic and X can be a one-atom or two-atom linkage.

Furthermore, hydroisomerization of the C₅-C₂₀₀ products, such as the C₅-C₁₀₀ products represented by Formula (VIII) or Formula (IX), such as C₂₅-C₅₀ polyolefin products, such as long-chain paraffins, can be performed using one or more suitable hydroisomerization catalyst(s) (e.g., MSDW-3™ catalyst; Pt/ZSM-48 catalyst; Pt/HZ SM-5 catalyst; Pt/HY catalyst; Pt/SAPO-1 1 catalyst). Hydroisomerization of the C₅-C₂₀₀ products, such as the C₅-C₁₀₀ products (VIII) can reduce the content of linear paraffins in hydrocarbon mixtures by producing branched compounds, and can enable the production of high-octane gasoline and low-pour-point diesel. The hydroisomerization of the C₅-C₂₀₀ products, such as the C₅-C₁₀₀ product represented by formula (VIII) can improve the viscosity properties of waxy feedstocks, for example.

In at least one embodiment, the hydroisomerization process is performed at a temperature of from about 15° C. to about 300° C., such as from about 30° C. to about 250° C., such as from about 50° C. to about 150° C., alternalty from about 50° C. to about 300° C.; at a pressure of from about 15 psi (103.42 kPa) to about 1000 psi (6,894.76 kPa), such as from about 30 psi (306.84 kPa) to about 500 psi (3,447.38 kPa); for a reaction time of from about 3 minutes to about 20 hours, such as from about 4 minutes to about 10 hours, such as from about 6 minutes to about 2 hours; and/or w weight hourly space velocity (WHSV) of from about 0.05 1h⁻¹ to about 20 h⁻¹, such as from about 0.1 h⁻¹ to about 15 h⁻¹, such as from about 0.5 h⁻¹ to about 10 h⁻¹.

### Lubricating Oils

Polyolefin products or hydrogenated polyolefin products of the present disclosure can be used as base stocks useful in engine oils. The polyolefin products and/or hydrogenated polyolefin products can be in the lube oil boiling range, such as from about 100° C. to about 450° C., for example.

The viscosity-temperature relationship of a lubricating oil is an aspect often considered when selecting a lubricant for a particular application. Viscosity index “VI” is an empirical, unitless number which indicates the rate of change in the viscosity of an oil within a given temperature range. Fluids exhibiting a relatively large change in viscosity with temperature are said to have a low viscosity index. A low VI oil, for example, will thin out at elevated temperatures faster than a high VI oil. For example, the high VI oil can be recommended because of its higher viscosity at higher temperature, which can be translated into thicker lubrication film and better protection of the contacting machine elements.

In another aspect, as the oil operating temperature decreases, the viscosity of a high VI oil will not increase as much as the viscosity of a low VI oil. This is advantageous because the high viscosity of the low VI oil will decrease the efficiency of the operating machine. Thus high VI (HVI) oil has performance advantages in both high and low temperature operation. VI is determined according to ASTM method D 2270. A lubricating oil of the present disclosure can have a VI of about 120 or greater, such as about 140 or greater, such as about 150 or greater, such as about 170 or greater, such as about 180 or greater, as determined according to ASTM D2270.

VI is related to kinematic viscosities measured at 40° C. and 100° C. using ASTM method D 445. A lubricating oil of the present disclosure can have a kinematic viscosity at 100° C. (KV100), as determined by ASTM D445, of about 2 cSt to about 25 cSt, such as from about 3 cSt to about 18 cSt, such as from about 4 cSt to about 10 cSt. Additionally or alternatively, a lubricating oil of the present disclosure can have a kinematic viscosity at 40° C. (KV40), as determined by ASTM D445, of about 10 cSt to about 125 cSt, such as from about 20 cSt to about 50 cSt.

Polyolefin products or hydrogenated polyolefin products of the present disclosure can be present in a lubricating oil in an amount of from about 1 wt % to about 99 wt %, such as from about 1 wt % to about 50 wt %, such as from about 1 wt % to about 25 wt %, such as from about 5 wt % to about 10 wt %, based on the weight of the lubricating oil.

### Other Lubricating Oil Additives

A lubricating oil of the present disclosure may additionally contain one or more lubricating oil performance additives including but not limited to dispersants, other detergents, corrosion inhibitors, rust inhibitors, metal deactivators, other anti-wear agents and/or extreme pressure additives, anti-seizure agents, wax modifiers, viscosity index improvers, viscosity modifiers, fluid-loss additives, seal compatibility agents, other friction modifiers, lubricity agents, anti-staining agents, chromophoric agents, defoamants, demulsifiers, emulsifiers, densifiers, wetting agents, gelling agents, tackiness agents, colorants, and others. For a review of suitable additives, see Klamann in Lubricants and Related Products, Verlag Chemie, Deerfield Beach, Fla.; ISBN 0-89573-177-0. Reference is also made to “Lubricant Additives Chemistry and Applications” edited by Leslie R. Rudnick, Marcel Dekker, Inc. New York, 2003 ISBN: 0-8247-0857-1.

Furthermore, the polyolefin products or hydrogenated polyolefin products of the present disclosure can be contacted/blended with one or more component(s), such as any fuel additives (e.g., metal deactivators, corrosion inhibitors, lead scavengers, fuel dyes, and antioxidant stabilizers), to form a biofuel composition. Examples of such one or more additive(s) may include anti-oxidants, corrosion inhibitors, ashless detergents, dehazers, dyes, lubricity improvers and/or mineral fuel components, but also conventional petroleum derived gasoline, diesel and/or kerosene. The amount of additive(s) can range from about 0.1 wt % to about 10 wt %, such as from about 0.5 wt % to about 8 wt %, such as from about 1 wt % to about 6 wt %, such as from about 2 wt % to about 4 wt %, based on the total weight of the polyolefin products or hydrogenated polyolefin products blend.

### Antioxidants

Suitable anti-oxidants may include phenolic anti-oxidants, aminic anti-oxidants and oil-soluble copper complexes. The phenolic antioxidants may include sulfurized and non-sulfurized phenolic antioxidants. The terms “phenolic type” or “phenolic antioxidant” used herein may include compounds having one or more than one hydroxyl group bound to an aromatic ring which may itself be mononuclear, e.g., benzyl, or poly-nuclear, e.g., naphthyl and spiro aromatic compounds. Thus “phenol type” may include phenol per se, catechol, resorcinol, hydroquinone, naphthol, etc., as well as alkyl or alkenyl and sulfurized alkyl or alkenyl derivatives thereof, and bisphenol type compounds including such bi-phenol compounds linked by alkylene bridges sulfuric bridges or oxygen bridges. Alkyl phenols may include mono- and poly-alkyl or alkenyl phenols, the alkyl or alkenyl group containing from about 3 carbons to about 100 carbons, such as from about 4 carbons to about 50 carbons, and sulfurized derivatives thereof, the number of alkyl or alkenyl groups present in the aromatic ring ranging from 1 to up to the available unsatisfied valences of the aromatic ring remaining after counting the number of hydroxyl groups bound to the aromatic ring.

A phenolic anti-oxidant may be represented by the general formula:

(R)ₓ—Ar—(OH)_{y}

where Ar is selected from phenyl, naphthyl, biphenyl,

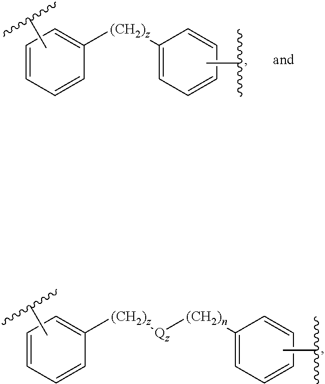

where R is a C₃-C₁₀₀ alkyl or alkenyl group, a sulfur substituted alkyl or alkenyl group, such as a C₄-C₅₀ alkyl or alkenyl group or sulfur substituted alkyl or alkenyl group, such as C₃-C₁₀₀ alkyl or sulfur substituted alkyl group, such as a C₄-C₅₀ alkyl group. Q is oxygen or sulfur. y is at least 1 to up to the available valences of Ar. x ranges from 0 to up to the available valances of Ar-y. z ranges from 1 to 10, n ranges from 0 to 20, and m is 0 to 4 and p is 0 or 1. In at least one embodiment, y ranges from 1 to 3, x ranges from 0 to 3, z ranges from 1 to 4 and n ranges from 0 to 5, and p is 0.

Phenolic anti-oxidant compounds can be the hindered phenolics and phenolic esters which include a sterically hindered hydroxyl group, and these include those derivatives of dihydroxy aryl compounds in which the hydroxyl groups are in the o- or p-position to each other. Suitable phenolic anti-oxidants include the hindered phenols substituted with C₁+ alkyl groups and the alkylene coupled derivatives of these hindered phenols. Examples of phenolic materials of this type 2-t-butyl-4-heptyl phenol; 2-t-butyl-4-octyl phenol; 2-t-butyl-4-dodecyl phenol; 2,6-di-t-butyl-4-heptyl phenol; 2,6-di-t-butyl-4-dodecyl phenol; 2-methyl-6-t-butyl-4-heptyl phenol; 2-methyl-6-t-butyl-4-dodecyl phenol; 2,6-di-t-butyl-4 methyl phenol; 2,6-di-t-butyl-4-ethyl phenol; and 2,6-di-t-butyl 4-alkoxy phenol; and

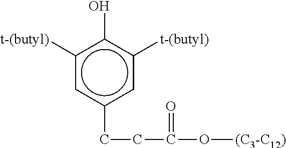

Phenolic type anti-oxidants in the lubricating industry may include commercial examples such as Ethanox® 4710, Irganox® 1076, Irganox® L1035, Irganox® 1010, Irganox® L109, Irganox® L118, Irganox® L135.

The phenolic anti-oxidant can be present in a lubricating oil in an amount in the range of from about 0.1 wt % to about 3 wt %, such as from about 1 wt % to about 3 wt %, such as from about 1.5 wt % to about 3 wt % based on the weight of the lubricant oil.

Aromatic amine anti-oxidants may include phenyl-α-naphthyl amine which is described by the following molecular structure:

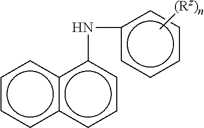

wherein R^{z} is hydrogen or a C₁ to C₁₄ linear or C₃ to C₁₄ branched alkyl group, such as C₁ to C₁₀ linear or C₃ to C₁₀ branched alkyl group, such as linear or branched C₆ to C₈ and n is an integer ranging from 1 to 5, such as 1. For example, an aromatic amine anti-oxidants can be Irganox® L06.

Other aromatic amine anti-oxidants may include other alkylated and non-alkylated aromatic amines, such as aromatic monoamines of the formula R¹⁹R²⁰R²¹N where R¹⁹ is an aliphatic, aromatic or substituted aromatic group, R²⁰ is an aromatic or a substituted aromatic group, and R²¹ is H, alkyl, aryl or R²²S(O)ₓR²³ where R²² is an alkylene, alkenylene, or aralkylene group, R²³ is a higher alkyl group, or an alkenyl, aryl, or alkaryl group, and x is 0, 1 or 2. The aliphatic group R⁸ may contain from 1 to 20 carbon atoms, or can contain from 6 to 12 carbon atoms. The aliphatic group is a saturated aliphatic group. For example, both R¹⁹ and R²⁰ are aromatic or substituted aromatic groups, and the aromatic group may be a fused ring aromatic group such as naphthyl. Aromatic groups R¹⁹ and R20 may be joined together with other groups such as S.

Suitable aromatic amine anti-oxidants have alkyl substituent groups of at least 6 carbon atoms. Examples of aliphatic groups may include hexyl, heptyl, octyl, nonyl, and decyl. For example, the aliphatic groups will not contain more than 14 carbon atoms. Suitable types of such other additional amine anti-oxidants which may be present include diphenylamines, phenothiazines, imidodibenzyls and diphenyl phenylene diamines. Mixtures of two or more of such other additional aromatic amines may also be present. Polymeric amine antioxidants can also be used.

Another class of anti-oxidant used in lubricating oil compositions and which may also be present are oil-soluble copper compounds. Any oil-soluble suitable copper compound may be blended into the lubricating oil. Examples of suitable copper antioxidants may include copper dihydrocarbyl thio- or dithio-phosphates and copper salts of carboxylic acid (naturally occurring or synthetic). Other suitable copper salts may include copper dithiacarbamates, sulphonates, phenates, and acetylacetonates. Basic, neutral, or acidic copper Cu(I) and or Cu(II) salts derived from alkenyl succinic acids or anhydrides may be particularly useful

Such anti-oxidants may be used individually or as mixtures of one or more types of anti-oxidants, the total amount used in a lubricating oil being an amount of from about 0.50 wt % to about 5 wt %, such as about 0.75 wt % to about 3 wt %.

### Detergents

Detergents may be included in lubricating oils of the present disclosure. In at least one embodiment, a detergent is an alkali or alkaline earth metal salicylate detergent.

A detergent can be alkali or alkaline earth metal phenates, sulfonates, carboxylates, phosphonates and mixtures thereof. The detergents can have total base number (TBN) ranging from neutral to highly overbased, e.g., TBN of 0 to 500 or greater, such as 2 to 400, such as 5 to 300, and they can be present either individually or in combination with each other in an amount in the range of from 0 wt % to about 10 wt %, such as from about 0.5 wt % to about 5 wt % (active ingredient) based on the total weight of the formulated lubricating oil.

Other detergents can be calcium phenates, calcium sulfonates, magnesium phenates, magnesium sulfonates and other related components (including borated detergents).

### Dispersants

During engine operation, oil-insoluble oxidation byproducts can be produced. Dispersants help keep these byproducts in solution, thus diminishing their deposition on metal surfaces. Dispersants may be ashless or ash-forming. For example, the dispersant is ashless. So called ashless dispersants are organic materials that form substantially no ash upon combustion. For example, non-metal-containing or borated metal-free dispersants can be considered ashless. In contrast, metal-containing detergents discussed above may form ash upon combustion.

Suitable dispersants may contain a polar group attached to a relatively high molecular weight hydrocarbon chain. The polar group may contain at least one nitrogen, oxygen, or phosphorus atom. Suitable hydrocarbon chains may contain from about 50 carbon atoms to about 400 carbon atoms.

In at least one embodiment, a dispersant is an alkenylsuccinic derivative, produced by the reaction of a long chain substituted alkenyl succinic compound, such as a substituted succinic anhydride, with a polyhydroxy or polyamino compound. The long chain group constituting the oleophilic portion of the molecule which confers solubility in the oil, can be a polyisobutylene group. Exemplary U.S. patents describing such dispersants are U.S. Pat. Nos. 3,172,892; 3,219,666; 3,316,177 and 4,234,435. Other types of dispersant are described in U.S. Pat. Nos. 3,036,003 and 5,705,458.

Hydrocarbyl-substituted succinic acid compounds may be used as dispersants, such as succinimide, succinate esters, or succinate ester amides prepared by the reaction of a hydrocarbon-substituted succinic acid compound, such as those having at least about 50 carbon atoms in the hydrocarbon substituent, with at least one equivalent of an alkylene amine.

Succinimides can be formed by the condensation reaction between alkenyl succinic anhydrides and amines. Molar ratios can vary depending on the amine or polyamine. For example, the molar ratio of alkenyl succinic anhydride to TEPA can vary from 1:1 to 5:1.

Succinate esters can be formed by the condensation reaction between alkenyl succinic anhydrides and alcohols or polyols. Molar ratios can vary depending on the alcohol or polyol used. For example, the condensation product of an alkenyl succinic anhydride and pentaerythritol can be used as a dispersant.

Succinate ester amides can be formed by condensation reaction between alkenyl succinic anhydrides and alkanol amines. For example, suitable alkanol amines may include ethoxylated polyalkylpolyamines, propoxylated polyalkylpolyamines and polyalkenylpolyamines such as polyethylene polyamines, such as propoxylated hexamethylenediamine.

The molecular weight of the alkenyl succinic anhydrides may range from about 800 g/mol to about 2,500 g/mol. The above products can be post-reacted with various reagents such as sulfur, oxygen, formaldehyde, carboxylic acids such as oleic acid, and boron compounds, such as borate esters or highly borated dispersants. The dispersants can be borated with from about 0.1 moles to about 5 moles of boron per mole of dispersant reaction product.

Mannich base dispersants can be made from the reaction of alkylphenols, formaldehyde, and amines. Process aids and catalysts, such as oleic acid and sulfonic acids, can also be part of the reaction mixture. Molecular weights of the alkylphenols may range from about 800 g/mol to about 2,500 g/mol.

High molecular weight aliphatic acid modified Mannich condensation products can be prepared from high molecular weight alkyl-substituted hydroxyaromatics or HN(R)₂ group-containing reactants. Examples of high molecular weight alkyl-substituted hydroxyaromatic compounds can be polypropylphenol, polybutylphenol, and other polyalkylphenols. These polyalkylphenols can be obtained by the alkylation, in the presence of an alkylating catalyst, such as BF₃, of phenol with high molecular weight polypropylene, polybutylene, and other polyalkylene compounds to give alkyl substituents on the benzene ring of phenol having an average molecular weight of from about 600 g/mol to about 100,000 g/mol.

Examples of HN(R)₂ group-containing reactants can be alkylene polyamines, such as polyethylene polyamines. Other representative organic compounds containing at least one HN(R)₂ group suitable for use in the preparation of Mannich condensation products may include a mono- and di-amino alkanes and their substituted analogs, e.g., ethylamine and diethanol amine; aromatic diamines, e.g., phenylene diamine, diamino naphthalenes; heterocyclic amines, e.g., morpholine, pyrrole, pyrrolidine, imidazole, imidazolidine, and piperidine; melamine and their substituted analogs.

Examples of alkylene polyamine reactants may include ethylenediamine, diethylene triamine, triethylene tetraamine, tetraethylene pentaamine, pentaethylene hexamine, hexaethylene heptaamine, heptaethylene octaamine, octaethylene nonaamine, nonaethylene decamine, and decaethylene undecamine and mixture of such amines having nitrogen contents corresponding to the alkylene polyamines, in the formula H₂N(JNH)aH, mentioned before, J is a divalent ethylene and a is 1 to 10 of the foregoing formula. Corresponding propylene polyamines such as propylene diamine and di-, tri-, tetra-, pentapropylene tri-, tetra-, penta- and hexaamines can be also suitable reactants. The alkylene polyamines can be obtained by the reaction of ammonia and dihalo alkanes, such as dichloro alkanes. Thus, the alkylene polyamines obtained from the reaction of 2 moles to 11 moles of ammonia with 1 mole to 10 moles of dichloroalkanes having 2 carbon atoms to 6 carbon atoms and the chlorines on different carbons can be suitable alkylene polyamine reactants.

Aldehyde reactants useful in the preparation of the high molecular products useful in this disclosure include the aliphatic aldehydes such as formaldehyde (also as paraformaldehyde and formalin), acetaldehyde and aldol (β-hydroxybutyraldehyde). Formaldehyde or a form aldehyde-yielding reactant is exemplary.

Dispersants can include borated and non-borated succinimides, including those derivatives from mono-succinimides, bis-succinimides, and/or mixtures of mono- and bis-succinimides, wherein the hydrocarbyl succinimide is derived from a hydrocarbylene group such as polyisobutylene having a molecular weight of from about 500 g/mol to about 5000 g/mol or derived from a mixture of such hydrocarbylene groups. Other exemplary dispersants may include succinic acid-esters and amides, alkylphenol-polyamine-coupled Mannich adducts, their capped derivatives, and other related components. Such additives may be used in an amount of from about 0.1 wt % to about 20 wt %, such as from about 0.1 wt % to about 8 wt %, such as from about 1 wt % to about 6 wt % (on an as-received basis) based on the weight of the total lubricant.

### Pour Point Depressants

Pour point depressants (also known as lube oil flow improvers) may also be present in lubricating oils of the present disclosure. Pour point depressant may be added to lower the minimum temperature at which the fluid will flow or can be poured. Examples of suitable pour point depressants include alkylated naphthalenes polymethacrylates, polyacrylates, polyarylamides, condensation products of haloparaffin waxes and aromatic compounds, vinyl carboxylate polymers, and terpolymers of dialkylfumarates, vinyl esters of fatty acids and allyl vinyl ethers. Such additives may be used in amount of from 0 wt % to about 0.5 wt %, such as from about 0.0001 wt % to about 0.3 wt %, such as from about 0.001 wt % to about 0.1 wt % based on the weight of the lubricating oil.

### Corrosion Inhibitors/Metal Deactivators

Corrosion inhibitors are used to reduce the degradation of metallic parts that are in contact with the lubricating oil composition. Suitable corrosion inhibitors may include aryl thiazines, alkyl substituted dimercapto thiodiazoles, thiadiazoles and mixtures thereof. Such additives may be used in an amount of from about 0.01 wt % to about 5 wt %, such as from about 0.01 wt % to about 1.5 wt %, such as from about 0.01 wt % to about 0.2 wt %, such as from about 0.01 wt % to about 0.1 wt %, based on the total weight of the lubricating oil.

### Seal Compatibility Additives

Seal compatibility agents help to swell elastomeric seals by causing a chemical reaction in the fluid or physical change in the elastomer. Suitable seal compatibility agents for lubricating oils may include organic phosphates, aromatic esters, aromatic hydrocarbons, esters (butylbenzyl phthalate, for example), and polybutenyl succinic anhydride and sulfolane-type seal swell agents such as Lubrizol® 730-type seal swell additives. Such additives may be used in an amount of from about 0.01 wt % to about 3 wt %, such as from about 0.01 wt % to about 2 wt %, based on the total weight of the lubricating oil.

### Anti-Foam Agents

Anti-foam agents may be included in lubricant oils of the present disclosure. These agents retard the formation of stable foams. Silicones and organic polymers can be anti-foam agents. For example, polysiloxanes, such as silicon oil or polydimethyl siloxane, provide antifoam properties. Anti-foam agents are commercially available and may be used in conventional minor amounts along with other additives such as demulsifiers; usually the amount of these additives combined is about 1 wt % or less, such as from about 0.001 wt % to about 0.5 wt %, such as from about 0.001 wt % to about 0.2 wt %, such as from about 0.0001 wt % to about 0.15 wt %, based on the total weight of the lubricating oil.

### Inhibitors and Antirust Additives

Anti-rust additives (or corrosion inhibitors) are additives that protect lubricated metal surfaces against chemical attack by water or other contaminants. One type of anti-rust additive can be a polar compound that wets the metal surface, protecting the metal surface with a film of oil. Another type of anti-rust additive can absorb water by incorporating it in a water-in-oil emulsion so that only the oil touches the metal surface. Yet another type of anti-rust additive chemically may adhere to the metal to produce a non-reactive surface. Examples of suitable additives may include zinc dithiophosphates, metal phenolates, basic metal sulfonates, fatty acids and amines. Other anti-wear additives may include zinc dithiocarbamates, molybdenum dialkyldithiophosphates, molybdenum dithiocarbamates, other organo molybdenum-nitrogen complexes, sulfurized olefins, etc. Such additives may be used in an amount of from about 0.01 wt % to about 5 wt %, such as from about 0.01 wt % to about 1.5 wt %, based on the total weight of the lubricating oil.

The term “organo molybdenum-nitrogen complexes” embraces the organo molybdenum-nitrogen complexes described in U.S. Pat. No. 4,889,647, which is incorporated by reference herein. The complexes can be reaction products of a fatty oil, dithanolamine and a molybdenum source. U.S. Pat. No. 4,889,647, which is incorporated by reference herein, reports an infrared spectrum for a reaction product of that disclosure; the spectrum identifies an ester carbonyl band at 1740 cm⁻¹ and an amide carbonyl band at 1620 cm⁻¹. The fatty oils can be glyceryl esters of higher fatty acids containing at least 12 carbon atoms up to 22 carbon atoms or more. The molybdenum source can be an oxygen-containing compound such as ammonium molybdates, molybdenum oxides and mixtures. Other organo molybdenum complexes can be tri-nuclear molybdenum-sulfur compounds described in EP 1,040,115 and WO 99/31113 and the molybdenum complexes described in U.S. Pat. No. 4,978,464, which are incorporated by reference herein.

### Diesel Fuels

In at least one embodiment, a diesel fuel is a C₅-C₂₀₀ hydrogenated polyolefin product, suhc as a C₅-C₁₀₀ hydrogenated polyolefin product, such as a C₆-C₂₅ hydrogenated polyolefin product.

The various types of carbon atoms of a polyolefin product of the present disclosure can be determined using ¹H NMR spectroscopy. For example, di-substituted olefin content and tri-substituted olefin content are indicators of linearity of a polyolefin product. A high amount of di-substituted olefin content indicates high linearity, and a low amount of tri-substituted olefin content indicates high linearity. In at least one embodiment, a polyolefin product has a di-substituted olefin content of from about 30% to about 80%, such as from about 50% to about 75%, such as from about 60% to about 70%, based on total unsaturations of the polyolefin product. A polyolefin product of the present disclosure can have a tri-substituted olefin content of less than 50%, based on total unsaturations of the polyolefin product. In at least one embodiment, a polyolefin product has a tri-substituted olefin content of from about 1% to about 50%, such as from about 5% to about 40%, such as from about 20% to about 40%, based on total unsaturations of the polyolefin product. The high linearity of polyolefin products of the present disclosure provides improved cetane number, as compared to highly branched polyolefin products.

Diesel engines may operate well with a cetane number of from 48 to 50. Fuels with a lower cetane number have longer ignition delays, requiring more time for the fuel combustion process to be completed. Hence, higher speed diesel engines operate more effectively with higher cetane number fuels. A hydrogenated polyolefin product of the present disclosure can be used as a diesel fuel, as indicated by advantageous cetane numbers. For example, a hydrogenated polyolefin product can have a cetane number of about 30 or greater, such as about 40 or greater, such as about 45 or greater, such as about 48 or greater, such as about 50 or greater, such as about 60 or greater, such as about 70 or greater, such as about 80 or greater, such as about 90 or greater.

### EXAMPLES

General considerations: All reagents and anhydrous solvents were purchased from Aldrich and Fisher Chemical, and were degassed, sparged with N₂ and dried over 3 521 molecular sieves prior to use. Deutrated solvents were purchased from Cambridge Isotope Laboratories and dried over molecular sieves prior to use. CuO was purchased as a nanopowder from Aldrich. Anhydrous cyclohexane was purchase from Aldrich. Adamantane was purchased from Aldrich. Grubbs 2^{nd} Generation Ru Catalyst was purchased from Strem Chemical. Solvents, polymerization grade toluene, C₃-C₅₀ cyclic alkanes and C₂-C₅₀ acyclic alkanes were supplied by ExxonMobil Chemical Company and thoroughly dried and degassed prior to use.

Gas Chromatography (GC): For the dehydrogenation of alkanes, the produts were analyzed using a GC (Agilent 6890 Plus) with an FID detector and a HP-PONA column (50 m length □ 0.2 mm diameter □ 0.5 □m film thickness). The GC conditions were the following: Injector: 225□C; 0.5 □L injection volume, 100/1 split ratio. Detector: 250□C. Oven: 35□C (10 min), 2.5□C/min to 135□C, 10□C/min to 320□C (6.5 min).

Spectra of products were recorded on a Bruker (400 MHz) spectrometer and referenced versus residual nondeuterated solvent shifts. The product samples were dissolved in chloroform-d or toluene-d⁸ in a 5-mm O.D. tube.

¹³C-NMR: Spectra of products were recorded on a Bruker (400 MHz) spectrometer and referenced versus residual nondeuterated solvent shifts. The product samples were dissolved in chloroform-d or toluene-d⁸ in a 5-mm O.D. tube.

Dehydrogenation of C₂-C₅₀ acyclic alkanes and C₃-C₅₀ cyclic alkanes in a heavy naphtha range.

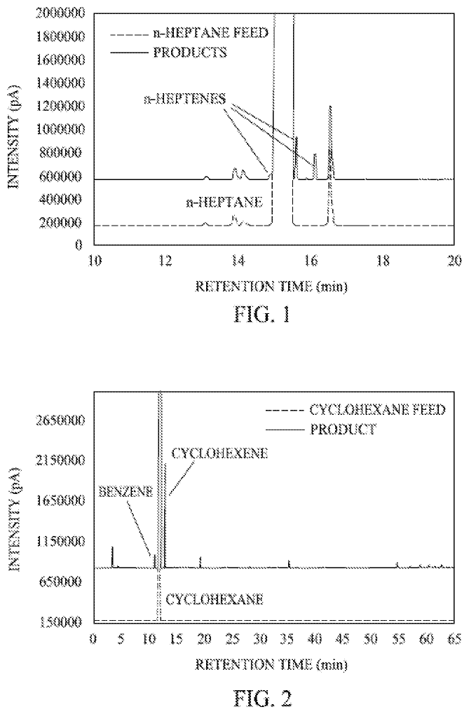

### Example 1 (FIG. 1). Dehydrogenation of n-heptane to Heptenes using CuO

In a N₂-filled glove-box, 0.5 g of CuO and 2.0 g of anhydrous n-heptane (Aldrich) were mixed in a 3 cm³ Swagelok stainless-steel pressure cell. The cell was sealed and placed in an oven held at 275° C. After 3 hours, the cell was taken out and allowed to cool down to room temperature. The cell was then opened and the liquid product recovered and analyzed by GC (FIG.1). Example 1 demonstrates that the dehydrogenation of n-heptane using CuO as a catalyst, led to the formation of heptenes, here obtained as primary products. The GC analysis is shown in FIG. 1.

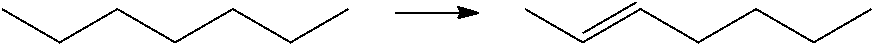

### Example 2 (FIG.2). Dehydrogenation of Cyclohexane to Cyclohexene using CuO

In an N₂-filled glove-box, 0.5 g of CuO and 2.0 g of anhydrous cyclohexane were mixed in a 3 cm³ Swagelok stainless-steel pressure cell. The cell was sealed and placed in an oven held at 275° C. After 3 hours, the cell was taken out and allowed to cool down to room temperature. The cell was then opened and the liquid product recovered and analyzed by GC (FIG. 2 ). Example 2 demonstrates that the dehydrogenation of cyclohexane using CuO as a catalyst, led to the formation of cyclohexene, here obtained as primary product. The GC analysis is shown in FIG. 2.

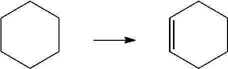 [SMILES: C1CCCCC1 ; C1=CCCCC1]

Ring-Opening Cross Metathesis (ROMP) of C₂-C₅₀ Acyclic and C₃-C₅₀ Cyclic Olefins (Examples 3-7).

In a 20 mL glass vial, a solution of selected olefin(s) (1 mL each), toluene (5 mL), adamantane (internal GC integration standard), and ring-opening cross metathesis catalyst were added. The ring-opening cross metathesis catalyst used in Examples 3-6 is Ru-Grubbs 2^{nd} Generation Catalyst (5 mg). The ring-opening cross metathesis catalyst used in Example 7 is heterogeneous Re catalyst (50 mg), activated with tetramethyl tin (3.5 mg). A heterogeneous Re catalyst was prepared by the incipient wetness impregnation method from NH₄ReO₄ and aluminum oxide and calcined at 550° C. for 3 hours. The results were analyzed by both GC and ¹H NMR.

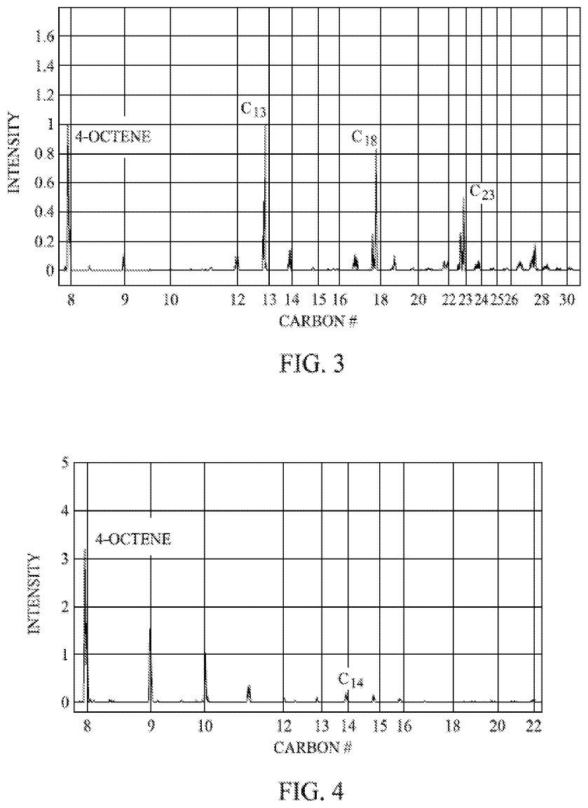

Example 3 (FIG. 3). Ring opening cross metathesis between cyclopentene and trans-4-octene. It was observed that with cyclopentene homo-metathesis, no oligomers were formed. However, only poly-cyclopentene was produced. The GC analysis demonstrates that the oligomers were formed when the two olefins were allowed to react with the Ru-Grubbs 2^{nnd} Generation catalyst

(FIG. 3), with a carbon number spacing of 5 carbons, thus due the cyclopentene ring.

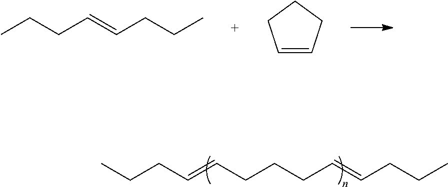

Example 4 (FIG. 4). Ring opening cross metathesis between cyclohexene and trans-4-octene. It was observed by GC analysis that the oligomers formed via ring-opening cross metathesis between cyclohexene and trans-4-octene were formed (FIG. 4). Under such reaction conditions, some olefin isomerization was observed. The major peak at C₁₄ is indicative of the coupling between the C₈ trans-4-octene and the C₆ cyclohexene.

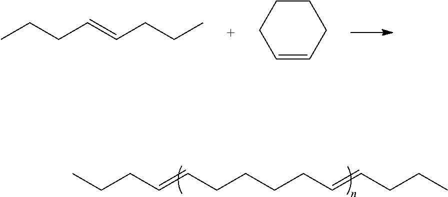

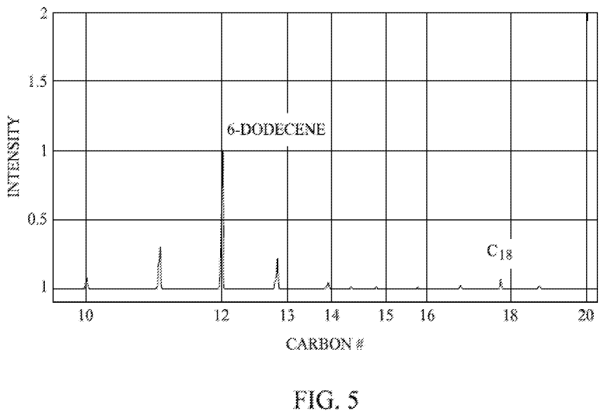

Example 5(FIG. 5). Ring opening cross metathesis between cyclohexene and 1-heptene. Analysis by both GC and ¹H NMR has shown that, in addition to the expected C₁₃ product from the ring-opening cross-metathesis of 1-heptene and cyclohexene, it was observed that 1-heptene can homocouple in order to give 6-dodecene. The dodecene (or two 1-heptenes) can then couple to give a C₁₈ product, which was demonstrated, and confirmed by GC analysis (FIG. 5).

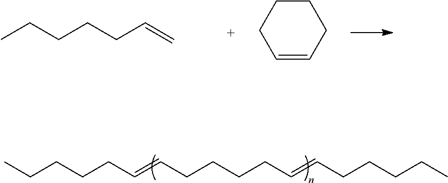

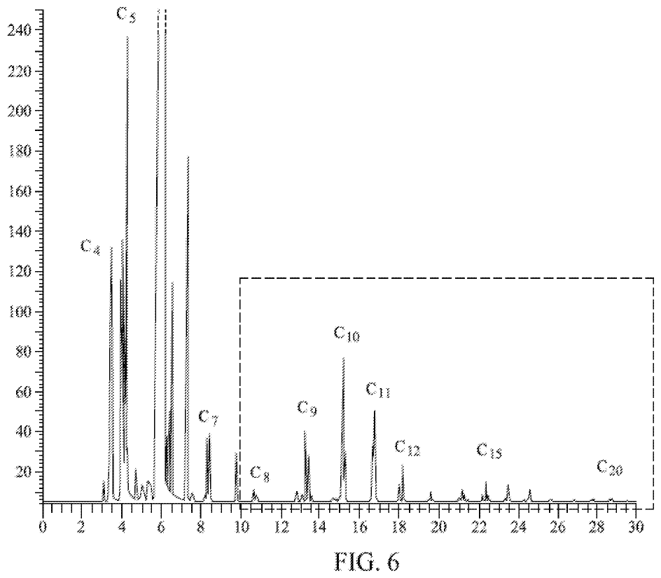

### Example 6 (FIG. 6). Ring Opening Cross Metathesis Between Mixed Feeds (e.g., Mixture of Pentenes) using Ru-Grubbs 2^{nd} Generation Catalyst

Mixed feeds, such as a mixture of C₅ olefins including cyclopentene, were tested with Ru-Grubbs 2^{nd} Generation Catalyst. The GC analysis of Example 6 is shown in FIG. 6, and demonstrates a distribution maxima at Cm and C_{15,} thus due to the cyclopentene ring opening metathesis.

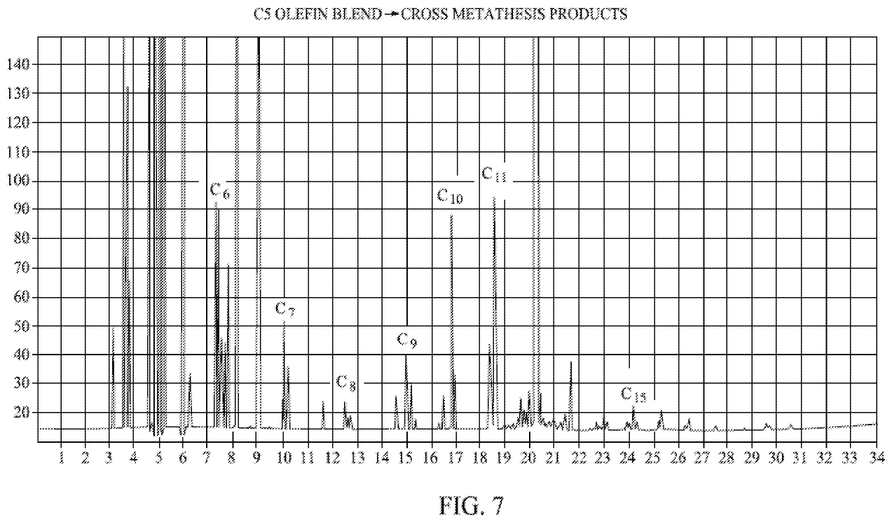

### Example 7 (FIG. 7). Ring Opening Cross Metathesis Between Mixed Feeds (e.g., Mixture of Pentenes) Using a Heterogeneous Re Catalyst

Mixed feeds, such as a mixture of C₅ olefins including cyclopentene, were also tested with a heterogeneous Re catalyst. Hence, the ring-opening cross metathesis reaction between a mixture of C₅ olefins including cyclopentene was studied, and the GC analysis of Example 7 is shown in FIG.7. The distribution maxima at C₁₀ and C₁₅ are due to the cyclopentene ring opening metathesis.

Hydrogenation and Hydroisomerization of the C₆-C₁₀₀ Olefins

The product from the metathesis reaction, 5.2 g, diluted with heptane was heated to 250° C. in the presence of 2 g of the MSDW-3™ catalyst under 300 psi (2,068.43 kPa) of hydrogen for 6 days.

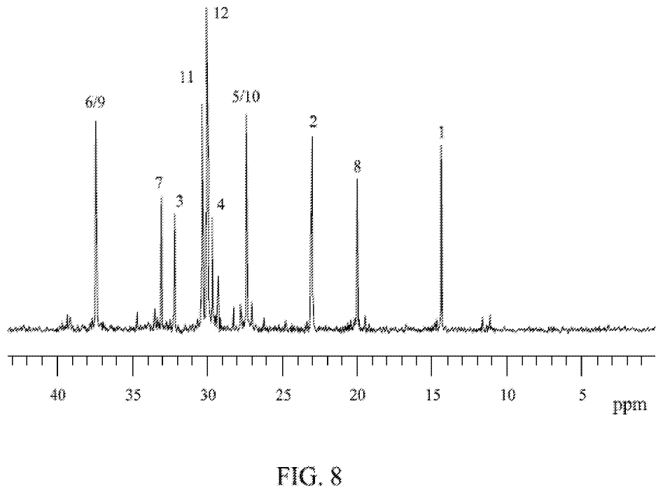

Product analysis was conducted after hydroisomerization was carried out with MSDW-3™ catalyst, to demonstrate the production of lubricant range products with branched structures, with methyl branches (Example 8, FIG. 8, peak 8). ¹³C-NMR spectra analysis of lubrication range products after hydroisomerization illustrates that the product displays a peak 1 at 6=14 ppm corresponding to the methyl terminus, and a peak 8 at δ=20 ppm, which correspond to the methyl branch.

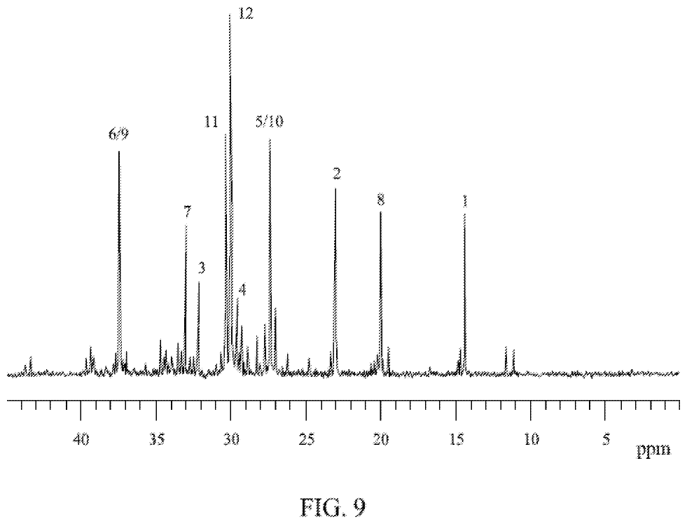

Hydrogenation of ring opening metathesis product of cyclooctene was carried out using H2 at 250° C., followed by the hydroisomerization using MSDW-3™ catalyst (Example 9, FIG. 9). ¹³C-NMR spectra analysis of lubrication range products after hydroisomerization between cyclooctene and 4-octene, illustrates that the product displays a peak 1 at 6=14 ppm corresponding to the methyl terminus, and a peak 8 at δ=20 ppm, which correspond to the methyl branch.

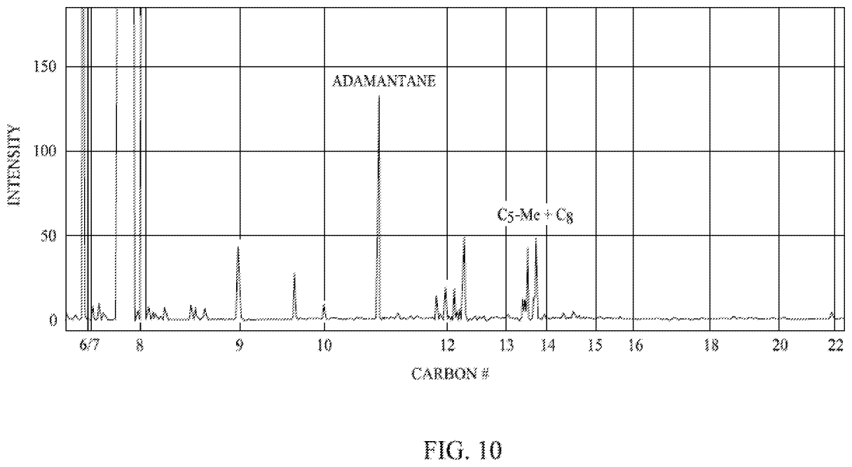

### Example 10 (FIG. 10). Substituted Cyclic Olefins: Synthesis of 5-methyltridecane Via Ring Opening Cross Metathesis Between methyl-cyclopentene and 4-octene, and Hydroisomerization

The ring-opening cross metathesis reaction between a methyl-cyclopentene and 4-octene was carried out using Grubbs 2nd generation catalyst, at 70° C. and ambient pressure, for 1 day, resulting to the formation of a methyl branched-C₁₄ polyolefin product. The methyl branched-C₁₄ polyolefin product was hydrogenated, and the peaks labeled c5-Me+C_{8,} were confirmed by GC-MS analysis (FIG. 10), illustrationg the production of 5-methyl-tridecane.

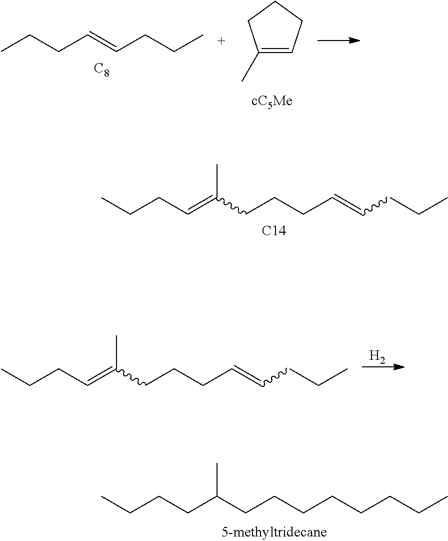

Overall, processes of the present disclosure provide C₆-C₁₀₀ polyolefin products including substituted and/or non-substituted olefins, from acyclic and cyclic olefins. Processes include converting hydrocarbons (such as heavy naphtha, including paraffins and/or naphthene-rich heavy virgin naphtha, such as C₂-C₅₀ cyclic and/or acyclic alkanes) to light distillates. The light distillate product includes the C₆-C₁₀₀ polyolefin products, which produce, for example, a fuel composition (e.g., higher value diesel (cetane)) or waxes, lubricant range products, or base stocks when blended with one or more other components (e.g., additives).

The phrases, unless otherwise specified, “consists essentially of” and “consisting essentially of” do not exclude the presence of other steps, elements, or materials, whether or not, specifically mentioned in this specification, so long as such steps, elements, or materials, do not affect the basic and novel characteristics of the present disclosure, additionally, they do not exclude impurities and variances normally associated with the elements and materials used.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, within a range includes every point or individual value between its end points even though not explicitly recited. Thus, every point or individual value may serve as its own lower or upper limit combined with any other point or individual value or any other lower or upper limit, to recite a range not explicitly recited.

All documents described herein are incorporated by reference herein, including any priority documents and/or testing procedures to the extent they are not inconsistent with this text. As is apparent from the foregoing general description and the specific embodiments, while forms of the present disclosure have been illustrated and described, various modifications can be made without departing from the spirit and scope of the present disclosure. Accordingly, it is not intended that the present disclosure be limited thereby. Likewise, the term “comprising” is considered synonymous with the term “including” for purposes of United States law. Likewise whenever a composition, an element or a group of elements is preceded with the transitional phrase “comprising,” it is understood that we also contemplate the same composition or group of elements with transitional phrases “consisting essentially of” “consisting of,” “selected from the group of consisting of” or “is” preceding the recitation of the composition, element, or elements and vice versa.

While the present disclosure has been described with respect to a number of embodiments and examples, those skilled in the art, having benefit of this disclosure, will appreciate that other embodiments can be devised which do not depart from the scope and spirit of the present disclosure.

1. A process for upgrading a hydrocarbon feed, comprising:
dehydrogenating a C₃-C₅₀ cyclic alkane and an C₂-C₅₀ acyclic alkane in the presence of a dehydrogenation catalyst to form a C₃-C₅₀ cyclic olefin and a C₂-C₅₀ acyclic olefin; and
introducing the C₃-C₅₀ cyclic olefin and the C₂-C₅₀ acyclic olefin to a group 6, 7 or 8 transition metal catalyst to form a C₅-C₂₀₀ olefin.

2. The process of claim 1, further comprising hydrogenating the C₅-C₂₀₀ olefin in the presence of a hydrogenation catalyst to form a C₅-C₂₀₀ hydrogenated product.

3. The process of claim 1, wherein the hydrocarbon feed is a naphtha feed comprising the C₃-C₅₀ cyclic alkane and the C₂-C₅₀ acyclic alkane to the catalyst.

4. The process of claim 3, wherein the naphtha feed further comprises one or more of n-hexane, n-heptane, cyclopentane, cyclohexane, methylcyclohexane, methylcyclopentane, benzene, toluene, xylenes, or a mixture thereof.

5. The process of claim 1, wherein the dehydrogenation catalyst is selected from CuO, Ag₂O, ZnO, NiO, CrOₓ, and VOₓ, FeOₓ, CoOₓ, MnOₓ, wherein x is in the range of 1 to 3.5.

6. The process of claim 1, wherein the C₂-C₅₀ acyclic alkane is ethane, propane, butane, pentane, hexane, heptane, octane, nonane, decane or mixtures thereof.

7. The process of claim 1, wherein the C₃-C₅₀ cyclic alkane is one or more of cyclopropane, cyclobutane, cyclopentane, cyclohexane, cycloheptane, cyclooctane, isomers, or mixtures thereof.

8. The process of claim 1, wherein a molar ratio of cyclic alkane to acyclic alkane is from about 1:250 to about 250:1.

9. The process of claim 8, wherein a molar ratio of cyclic alkane to acyclic alkane is from about 1:10 to about 10:1.

10. The process of claim 1, wherein dehydrogenating is performed:
at a temperature of about 150° C. to about 350° C.; and/or at a pressure of from about 1 bar gauge to about 750 bar gauge.

11. The process of claim 1, wherein dehydrogenating is performed:
at a temperature higher than 400° C.; and/or
at a pressure of from about less than 1 bar gauge to about 2 bar gauge.

12. The process of claim 1, wherein the dehydrogenation catalyst is present at a catalyst loading % (based on the concentration of alkanes) of from about 0.5 mol % to about 5 mol %.

13. The process of claim 1, wherein the group 6, 7 or 8 transition metal catalyst is a group 6 catalyst that is a molybdenum-containing catalyst.

14. The process of claim 1, wherein the group 6, 7 or 8 transition metal catalyst is a group 7 catalyst that is a rhenium-containing catalyst.

15. The process of claim 1, wherein the group 6, 7 or 8 transition metal catalyst is a group 8 catalyst that is a ruthenium-containing catalyst.

16. The process of claim 1, wherein reacting the C₃-C₅₀ cyclic olefin and the C₂-C₅₀ acyclic olefin is performed:
at a temperature from about 25° C. to about 450° C.; and/or
at a pressure of from about 100 kPa to about 2,000kPa.

17. The process of claim 1, wherein reacting the C₃-C₅₀ cyclic olefin and the C₂-C₅₀ acyclic olefin is performed at a catalyst loading of from about 0.01 mol % to about 10 mol %.

18. The process of claim 1, wherein the hydrogenation catalyst is a Raney nickel catalyst or a palladium catalyst supported on activated carbon.

19. The process of claim 1, wherein hydrogenating is performed at:
a pressure of from about 4,500 KPa to about 8,000 KPa; and/or
at a temperature of from about 30° C. to about 400° C.

20. The process of claim 1, wherein hydrogenating is performed at a pressure of hydrogen of from 200 psi (1,378.95 kPa) to about 400 psi (2,757.9 kPa).

21. The process of claim 1, wherein hydrogenating is performed at a molar ratio of H₂ to C₆-C₂₀₀ olefin of from about 1000:1 to about 100:1.

22. The process of claim 1, further comprising hydroisomerizing the C₅-C₂₀₀ hydrogenated product in the presence of a hydroisomerization catalyst to form a C₅-C₂₀₀ hydroisomerized product.

23. The process of claim 22, wherein hydroisomerizing is performed:
at a temperature of from about 50° C. to about 300° C.;
at a pressure of from about 30 psi (206.84 kPa) to about 500 psi (3,447.38 kPa); and/or
a weight hourly space velocity (WHSV) of from about 0.5 h⁻¹ to about 10 h⁻¹.

24. The process of of claim 1, wherein the cyclic olefin is represented by Formula (II): 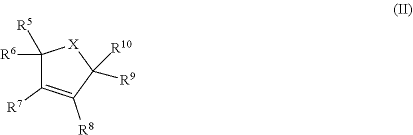
wherein:
X is a one-atom to five-atom linkage;
one of R⁷ and R⁸ is hydrogen and the other is selected from hydrogen, hydrocarbyl, substituted hydrocarbyl, heteroatom-containing hydrocarbyl, and substituted heteroatom-containing hydrocarbyl; and
R⁵, R⁶, R⁹, and R¹⁰ are independently selected from hydrogen, hydrocarbyl, substituted hydrocarbyl, heteroatom-containing hydrocarbyl, and substituted heteroatom-containing hydrocarbyl, or two or more of R⁵, R⁶, R⁹, and R¹⁰ can be taken together to form a cyclic group.

25. The process of claim 1, wherein the C₂-C₅₀ acyclic olefin is represented by Formula (I): 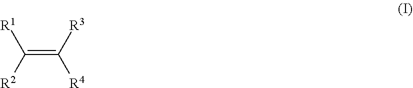
wherein:
R¹, R², R³, and R⁴ are independently selected from hydrogen, C₁-C₄₀ hydrocarbyl, C₁-C₄₀ substituted hydrocarbyl, a heteroatom, and a heteroatom-containing group.

26. The process of claim 1, wherein the C₅-C₂₀₀ olefin is represented by Formula (III): 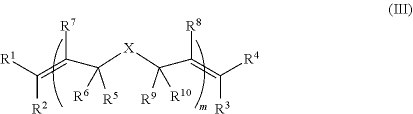
wherein:
X is a one-atom to five-atom linkage;
m is 1 to 50;
R¹, R², R³, and R⁴ are independently selected from hydrogen, C₁-C₄₀ hydrocarbyl, C₁-C₄₀ substituted hydrocarbyl, a heteroatom, and a heteroatom-containing group;
R⁵, R⁶, R⁹, and R¹⁰ are independently selected from hydrogen, hydrocarbyl, substituted hydrocarbyl, heteroatom-containing hydrocarbyl, and substituted heteroatom-containing hydrocarbyl, or two of R⁵, R⁶, R⁹, and R¹⁰ may be taken together to form a cyclic structure; and
one of R⁷ and R⁸ is hydrogen and the other is selected from hydrogen, hydrocarbyl, substituted hydrocarbyl, heteroatom-containing hydrocarbyl, and substituted heteroatom-containing hydrocarbyl.

27. The process of claim 1, wherein the C₅-C₂₀₀ hydrogenated product is represented by Formula (VIII): 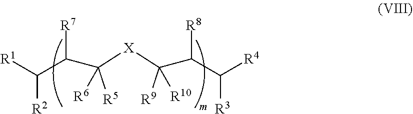
wherein:
X is a one-atom to five-atom linkage;
m is 1 to 50;
R¹, R², R³, and R⁴ are independently selected from hydrogen, C₁-C₄₀ hydrocarbyl, C₁-C₄₀ substituted hydrocarbyl, a heteroatom, and a heteroatom-containing group;
one of R⁷ and R⁸ is hydrogen and the other is selected from hydrogen, hydrocarbyl, substituted hydrocarbyl, heteroatom-containing hydrocarbyl, and substituted heteroatom-containing hydrocarbyl; and
R⁵, R⁶, R⁹, and R¹⁰ are independently selected from hydrogen, hydrocarbyl, substituted hydrocarbyl, heteroatom-containing hydrocarbyl, and substituted heteroatom-containing hydrocarbyl, or two of R⁵, R⁶, R⁹, and R¹⁰ may be taken together to form a cyclic structure.
